(19) United States
(12) Patent Application Publication
(10) Pub. No.: US 2025/0040985 A1
(43) Pub. Date: Feb. 6, 2025

(54) LESION OPTIMIZATION IN THE USE OF PULSED ELECTRIC FIELDS

(71) Applicant: Galvanize Therapeutics, Inc., Redwood City, CA (US)

(72) Inventors: Steven D. GIROUARD, Redwood City, CA (US); Jacob LAUGHNER, Redwood City, CA (US); Armaan VACHANI, Redwood City, CA (US); Quim CASTELLVI, Redwood City, CA (US); Robert E. NEAL, II, Redwood City, CA (US); Curt R. EYSTER, Redwood City, CA (US)

(73) Assignee: Galvanize Therapeutics, Inc., Redwood City, CA (US)

(21) Appl. No.: 18/923,425

(22) Filed: Oct. 22, 2024

Related U.S. Application Data
(63) Continuation of application No. PCT/US23/20282, filed on Apr. 27, 2023.
(60) Provisional application No. 63/336,197, filed on Apr. 28, 2022.

(51) Int. Cl. A61B 18/14; A61B 18/00

(52) U.S. Cl. CPC A61B 18/1492; A61B 2018/00357; A61B 2018/00577

(57) ABSTRACT

Devices, systems and methods are provided for treating a variety of conditions with PEF energy. PEF energy is typically characterized as high voltage pulsed energy that is configured to be delivered in one or more doses. The one or more doses create a lesion in the tissue. Lesion dimensions vary depending on the parameters of the PEF waveform and the dose, among other influences such as tissue type, etc. Therefore, different waveforms of PEF energy and different doses of PEF energy provide different results, particularly different lesion sizes and types. Systems, devices, and methods are provided that allow a physician or user to control the PEF energy delivered to provide a desired result and/or provide predictive information with which the user can utilize to generate a desired result.

### CROSS REFERENCE TO RELATED APPLICATIONS

This application is a continuation of PCT Application No. US2023/020282, filed Apr. 27, 2023, which claims the benefit of U.S. Provisional Application No. 63/336,197, filed Apr. 28, 2022, the content of each of which is incorporated herein by reference in their entirety.

### BACKGROUND

Pulsed electric field (PEF) therapies use brief applications of electrical energy to the body for the treatment of diseases and afflictions. In some instances, such delivery is to tissues within a body lumen, passageway or similar anatomy, or reachable endoluminally through such passageways. Such devices typically include a flexible elongate shaft, so as to traverse tortuous luminal anatomy, and an energy delivery element mounted thereon to deliver such energy to remote or enclosed locations such as body lumens. Such devices have been developed to treat, for example, passageways of the lungs or blood vessels of the vasculature, or to treat various organs such as the heart, stomach, intestines, etc. In other instances, delivery is direct through open surgery or through a percutaneous approach. Different environments, such as airways versus blood filled environments, and differing diseases, such as those affecting the surface tissues versus those affecting deeper layers or tissues, lead to varying designs for these devices.

The PEF energy disrupts the integrity of the target tissue cells which initiates a cascade of biochemical processes which induce different forms of cell death including necrosis, apoptosis, aponecrosis, necroptosis, and/or pyroptosis. Since PEF therapies are not dependent on thermal processes, cells are killed within a volume of tissue in-vivo without altering the stromal proteins and extracellular matrix within that volume and thus facilitating the preserved function of those critical and sensitive anatomic structures, such as the major vasculature, luminal systems such as the common bile duct, and tissue structures such as the pleura. Thus, PEF therapies offer a superior safety profile relative to other ablative modalities. As a consequence of those characteristics, PEF is being applied in a variety of different disease states with increasing regularity, including cancer, heart disease, and lung disease.

Variations exist between clinical PEF systems, including differences in waveform parameters and delivery polarity (i.e., bipolar or monopolar). In bipolar electrode configurations, energy is delivered between effector devices placed within, or adjacent to, the targeted environment. Conversely, monopolar systems deliver energy from effector devices placed within, or adjacent to, the targeted environment to a remote dispersive electrode serving as the electrical return. The dispersive electrode is of sufficient surface area to distribute the PEF energy broadly enough that no treatment effects are encountered at its location. Traditionally, radiofrequency (RF) energy has been used to treat a variety of ailments, including cardiovascular conditions. Such energy can be applied to the heart and vasculature for the treatment of a variety of conditions, including atherosclerosis (particularly in the prevention of restenosis following angioplasty) and atrial fibrillation. Atrial fibrillation is the most common sustained cardiac arrhythmia, and severely increases the risk of mortality in affected patients, particularly by causing stroke. In this phenomenon, the heart is taken out of normal sinus rhythm due to the production of erroneous electrical impulses. Atrial fibrillation is thought to be initiated in the myocardial sleeves of the pulmonary veins (PVs) due to the presence of automaticity in cells within the myocardial tissue of the PVs. Pacemaker activity from these cells is thought to result in the formation of ectopic beats that initiate atrial fibrillation. PVs are also thought to be important in the maintenance of atrial fibrillation because the chaotic architecture and electrophysiological properties of these vessels provides an environment where atrial fibrillation can be perpetuated. Thus, destruction or removal of these aberrant pacemaker cells within the myocardial sleeves of the PVs has been a goal and atrial fibrillation is often treated by delivering therapeutic energy to the pulmonary veins. However, due to reports of PV stenosis, the approach has been conventionally modified to one that targets PV antra to achieve conduction block between the PVs and the left atrium. The PV antra encompass, in addition to the pulmonary veins, the left atrial roof and posterior wall and, in the case of the right pulmonary vein antra, a portion of the interatrial septum. In some instances, this technique offers a higher success rate and a lower complication rate compared with pulmonary vein ostial isolation.

Thermal ablation therapies, especially radiofrequency (RF) ablation, are currently the “gold standard” to treat symptomatic atrial fibrillation by localized tissue necrosis. Typically, RF ablation is used to create a ring of ablation lesions around the outside of the ostium of each of the four pulmonary veins. RF current causes desiccation of tissue by creating a localized area of heat that results in discrete coagulation necrosis. The necrosed tissue acts as a conduction block thereby electrically isolating the veins.

Despite the improvements in reestablishing sinus rhythm using available methods, both success rate and safety are limited by the thermal nature of these procedures. Complications include pulmonary vein stenosis, phrenic nerve injury, esophageal injury, atrio-esophageal fistula, peri-esophageal vagal injury, perforations, thromboembolic events, vascular complications, and acute coronary artery occlusion, to name a few. Thus, while keeping the technique in clinical practice, safer and more versatile methods of removing abnormal tissue have been used, such as pulsed electric field therapy (PEF). However, since PEF therapy has a different cellular effect on the tissue, the tissue reacts differently than when receiving RF ablation. Consequently, the conventional cues that physicians monitor throughout an ablation procedure are largely inapplicable and can lead to inferior results, such as an incomplete block of the aberrant electrical rhythms. In addition, the different effect of the PEF energy on tissue causes different lesion sizes than RF and a variety of other differences that are unanticipated by the physician. Therefore, improvements in methods, devices and systems are desired that improve the usability of PEF systems and the resultant outcomes of treatment. Such treatments should be safe, effective, and lead to reduced complications. At least some of these objectives will be met by the systems, devices and methods described herein.

### SUMMARY

Described herein are embodiments of apparatuses, systems and methods for treating target tissue, particularly cardiac tissue. Likewise, the invention relates to the following numbered clauses:
1. A system for generating a lesion in tissue comprising:
   a treatment catheter having at least one delivery electrode; and
   a generator electrically couplable to the treatment catheter, wherein the generator includes a processor that generates at least one algorithm configured to provide an electric signal of pulsed electric field energy deliverable through the delivery electrode, wherein the processor generates the algorithm based on at least one of one or more inputs by a user.
2. A system as in clause 1, wherein the processor dynamically generates the algorithm based on the one or more inputs in real time.
3. A system as in clause 1, wherein the processor selects the algorithm based on the one or more inputs from a group of algorithms.
4. A system as in any of the above clauses, wherein the one or more inputs comprises a desired dimension of the lesion.
5. A system as in any of the above clauses, wherein the one or more inputs comprises a threshold for a temperature rise.
6. A system as in any of the above clauses, wherein the one or more inputs comprises a threshold for muscle contraction.
7. A system as in any of the above clauses, wherein the one or more inputs comprises a threshold dose time.
8. A system as in any of the above clauses, wherein the one or more inputs comprises a plurality of the one or more inputs.
9. A system as in clause 8, wherein the at least one of the one or more inputs comprises a plurality of the one or more inputs.
10. A system as in clause 8, wherein the processor utilizes the plurality of the one or more inputs and based on a ranking or weighting of the plurality of the one or more inputs.
11. A system as in any of the above clauses, wherein the processor utilizes the one or more inputs and at least one default value to generate the algorithm.
12. A system as in any of the above clauses, wherein at least one of the one or more inputs is selectable by the user from a menu.
13. A system as in any of the above clauses, wherein at least one of the one or more inputs is programmable by the user with a user interface.
14. A system as in any of the above clauses, wherein the processor correlates the one or more inputs to electric field strength thresholds.
15. A system as in clause 14, wherein the processor determines the electric field strengths of the at least one delivery electrode.
16. A system as in clause 15, wherein the processor correlates the electric field strengths to the electric field strength thresholds.
17. A system as in any of the above clauses, wherein the processor selects at least one parameter of the at least one algorithm to generate the electric signal.
18. A system as in any of the above clauses, wherein the processor modifies the algorithm based on tissue thickness.
19. A system as in clause 18, wherein tissue thickness is determined by ultrasound, magnetic resonance or fluoroscopy.
20. A system for providing a treatment to tissue comprising:
   a treatment catheter having at least one delivery electrode; and
   a generator electrically couplable to the treatment catheter, wherein the generator includes a processor that provides at least one algorithm configured to provide an electric signal of pulsed electric field energy deliverable through the delivery electrode to provide the treatment, wherein the processor generates a prediction of an outcome of the treatment based on at least one of one or more inputs by a user.
21. A system as in clause 20, wherein the prediction comprises at least one lesion dimension, temperature rise, muscle contraction, and/or dose time.
22. A system as in clause 21, wherein the at least one lesion dimension comprises lesion depth.
23. A system as in any of clauses 21-22, wherein the at least one lesion dimension comprises lesion width.
24. A system as in any of clauses 20-23, wherein the one or more inputs comprises a threshold for a temperature rise.
25. A system as in any of clauses 20-24, wherein the one or more inputs comprises a threshold for muscle contraction.
26. A system as in any of clauses 20-25, wherein the one or more inputs comprises a threshold dose time.
27. A system as in any of clauses 20-26, wherein the one or more inputs comprises a plurality of inputs.
28. A system as in clause 27, wherein the at least one of the one or more inputs comprises a plurality of the one or more inputs.
29. A system as in in any of clauses 20-28, wherein at least one of the one or more inputs is selectable by the user from a menu.
30. A system as in in any of clauses 20-29, wherein at least one of the one or more inputs is programmable by the user with a user interface.
31. A system as in clause 20, wherein the one or more inputs comprises a single input.
32. A system as in clause 31, wherein the single input comprises dose time.
33. A system for generating a lesion in tissue comprising:
   a treatment catheter having at least one delivery electrode; and
   a generator electrically couplable to the treatment catheter, wherein the generator includes a processor that provides at least one algorithm configured to provide an electric signal of pulsed electric field energy deliverable through the delivery electrode to provide the treatment, wherein the processor generates a spacing distance recommendation between the lesion and a next lesion.
34. A system as in clause 33, wherein the tissue comprises cardiac tissue and the delivery electrode is configured to deliver the energy to a surface of the cardiac tissue in a manner so as to create a parabolic lesion.
35. A system as in clause 34, wherein the parabolic lesion has a stun zone.
36. A system as in clause 35, wherein the spacing distance recommendation positions the next lesion sufficiently close to the lesion so that the stun zone at least partially overlaps with the next lesion.
37. A system as in clause 36, wherein the next lesion has a stun zone and wherein the spacing distance recommendation at least overlaps the stun zone of the lesion with the stun zone of the next lesion so that the tissue between the centers of the lesions form a continuous permanent lesion.
38. A system as in any of clauses 33-37, wherein processor generates the spacing distance recommendation between the lesion and a next lesion dynamically based on local electrophysiologic information.
39. A system as in clause 38, wherein the local electrophysiologic information is obtained from intra-procedure voltage maps and post application electrogram voltage changes.
40. A system as in clause 38, wherein the local electrophysiologic information is obtained through comprises an observed voltage reduction in the tissue in response to energy delivery.
41. A system as in any of clauses 33-40, wherein the processor is configured to modify the spacing distance recommendation based on tissue thickness.
42. A system as in clause 41, wherein the tissue thickness is determined by ultrasound, magnetic resonance or fluoroscopy.
43. A system as in any of clauses 33-41, further comprising an electroanatomic mapping system, and wherein the spacing distance recommendation is visually displayed by the electroanatomic mapping system.
44. A system for generating a lesion in tissue comprising:
   a generator electrically couplable to a treatment catheter, wherein the generator includes a processor that generates at least one algorithm configured to provide an electric signal of pulsed electric field energy deliverable through a delivery electrode of the treatment catheter, wherein the processor generates the algorithm based on at least one of one or more inputs by a user.
45. A system for providing a treatment to tissue comprising:
   a generator electrically couplable to a treatment catheter, wherein the generator includes a processor that provides at least one algorithm configured to provide an electric signal of pulsed electric field energy deliverable through a delivery electrode of the treatment catheter to provide the treatment, wherein the processor generates a prediction of an outcome of the treatment based on at least one of one or more inputs by a user.
46. A system for generating a lesion in tissue comprising:
   a generator electrically couplable to a treatment catheter, wherein the generator includes a processor that provides at least one algorithm configured to provide an electric signal of pulsed electric field energy deliverable through a delivery electrode the treatment catheter to provide the treatment, wherein the processor generates a spacing distance recommendation between the lesion and a next lesion.

These and other embodiments are described in further detail in the following description related to the appended drawing figures.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an embodiment of a tissue modification system.

FIGS. 2A-2B illustrates embodiments of a treatment catheter configured to deliver focal therapy.

FIG. 3 illustrates an embodiment of a waveform of a signal prescribed by an energy delivery algorithm.

FIG. 4 illustrates a portion of the heart showing a cut-away of the right atrium and left atrium with a treatment catheter positioned therein.

FIG. 5 illustrates an embodiment of a waveform of a signal prescribed by an energy delivery algorithm.

FIG. 6 is a schematic illustration of a lesion generated by a catheter, such as illustrated in FIG. 2B.

FIG. 7 illustrates an example of three different levels or doses of energy delivery.

FIG. 8 illustrates the generation of two adjacent lesions that are overlapping.

FIG. 9 illustrates the resultant continuous lesion formed from the energy delivered by the catheter.

FIG. 10 illustrates the effects of multiple doses on lesion size.

FIG. 11 illustrates an effect of overlapping lesions.

FIG. 12 illustrates a zone that receives double dosing from both catheter placements and is considered part of the treatment depth of the continuous lesion.

FIG. 13 illustrates a resulting continuous lesion having treatment depth that increased in comparison to FIG. 8.

FIG. 14 illustrates lesion spacing that is larger than that of FIG. 11 and a zone that receives double dosing from both catheter placements that has shifted upwards.

FIG. 15 illustrates a continuous lesion as a result of the lesions of FIG. 14.

FIG. 16 illustrates example correlations between treatment depth and lesion spacing at three different energy doses.

FIGS. 17A-17B illustrate the use of two different treatment protocols for different areas of the heart.

FIG. 18 illustrates example placements of anterior and posterior lesions.

FIG. 19 illustrates pulmonary vein isolation rates at a 90-day remap.

FIG. 20 illustrates a system having user inputs which are used by the system to generate or select a particular energy delivery algorithm that optimizes the treatment waveform and/or dose for the desired treatment.

FIGS. 21A-21D illustrate example individual relationships between treatment/lesion depth and the parameters of packet number, frequency, packet delivery rate and packet duration.

FIGS. 22A-22D illustrate example individual relationships between treatment/lesion width and the parameters of packet number, frequency, packet delivery rate and packet duration.

FIGS. 23A-23D illustrate example individual relationships between treatment/lesion width and the parameters of packet number, frequency, packet delivery rate and temperature rise.

FIGS. 24A-24D illustrate example individual relationships between treatment/lesion width and the parameters of packet number, frequency, packet delivery rate and muscle contraction.

FIGS. 25A-25C illustrate example electric field distributions for a catheter embodiment delivering PEF energy within the heart.

FIG. 26 illustrates a system that provides a prediction of lesion dimensions.

FIG. 27 illustrates a system that provides a prediction of lesion dimensions based on desired dose time.

### DETAILED DESCRIPTION

Devices, systems and methods are provided for treating a variety of conditions with PEF energy. PEF energy is typically characterized as high voltage pulsed energy that is configured to be delivered in one or more doses. Each energy dose delivered to the target tissue is configured to maintain the temperature at or in the target tissue below a threshold for thermal ablation. Instead of inducing damage from thermal ablation, described as extracellular protein coagulation, the effects are considered non-thermal wherein such energy modifies or destroys cells within the tissue but preserves the underlying protein extracellular matrix of tissues that provides the interstitial architectural structure and structure-related functions of the tissue. In some instances, this allows regeneration of tissue, such as by repopulation of the extracellular matrices. In addition, nearby sensitive tissues are spared injury. It may be appreciated that the doses may be titrated or moderated over time so as to further reduce or eliminate thermal buildup during the treatment procedure. It may be appreciated that in some embodiments energy delivery is actuated by a variety of mechanisms, such as with the use of an actuator on the device or a foot switch operatively connected to a generator. Such actuation typically provides a single energy dose or activation.

Target tissue cells may be treated in any location throughout the body, including cells of the digestive system (e.g. mouth, glands, esophagus, stomach, duodenum, jejunum, ileum, intestines, colon, rectum, liver, gall bladder, pancreas, anal canal, etc.), cells of the respiratory system (e.g. nasal cavity, pharynx larynx, trachea, bronchi, lungs, etc.), cells of the urinary system (e.g. kidneys, ureter, bladder, urethra, etc.), cells of the reproductive system (e.g. reproductive organs, ovaries, fallopian tubes, uterus, cervix, vagina, testes, epididymis, vas deferens, seminal vesicles, prostate, glands, penis, scrotum, etc.), cells of the endocrine system (e.g. pituitary gland, pineal gland, thyroid gland, parathyroid gland, adrenal gland), cells of the circulatory system (e.g. heart, arteries, veins, etc.), cells of the lymphatic system (e.g. lymph node, bone marrow, thymus, spleen, etc.), cells of the nervous system (e.g. brain, spinal cord, nerves, ganglia, etc.), cells of the eye (e.g. retina, macula, Layer of Rods and Cones, retinal pigment epithelium optic nerve, choroid, sclera, etc.), cells of the muscular system (e.g. myocytes, etc.), and cells of the skin (e.g. epidermis, dermis, hypodermis, etc.), to name a few.

Conditions treated include arrhythmias. When treating atrial fibrillation, closed paths of contiguous ablation lesions, such as around the outside ostium of a pulmonary vein, are typically created by the delivery of PEF energy with either focal catheters or single shot catheters. In addition, the focal catheters can be used to create many other types of lesions, particularly lines along various surfaces of cardiac tissue. In one embodiment, a cavo-tricuspid isthmus line is created for the treatment of typical atrial flutter in the right atrium. In another embodiment, roof lines and/or floor lines are created for a box lesion along the posterior wall of the left atrium for patients with atrial fibrillation, particularly for persistent atrial fibrillation. In another embodiment, a mitral isthmus line is created along the anterior or lateral wall of the left atrium for atypical atrial flutter. In yet another embodiment, ventricular lines are created connecting two inexcitable boundaries that are critical to the initiation or maintenance of a reentrant ventricular arrhythmia, typically in patients with ventricular tachycardia resulting from ischemic heart disease. Other conditions treated include pulmonary disorders, such as chronic obstructive pulmonary disease, chronic bronchitis, mucus hypersecretion, asthma and cystic fibrosis, to name a few. Still other conditions comprise a coagulation disorder. In some embodiments, the disorder comprises a neurological disorder, e.g., an injury or disease. In some embodiments, the disorder comprises a cardiovascular disorder, such as a degenerative heart disease, a heart failure disease, a coronary artery disease, an ischemia, angina pectoris, an acute coronary syndrome, a peripheral vascular disease, a peripheral arterial disease, a cerebrovascular disease, or atherosclerosis. In some embodiments, the disorder comprises an immune disorder, e.g., an autoimmune disorder. In some embodiments, the disorder comprises a liver disease. In some embodiments, the disorder comprises a tumor or cancer, such as a blood cancer or a solid tissue cancer.

The devices, systems and methods deliver therapeutic energy to tissue in the body to provide tissue modification, typically to create a lesion. The lesion is the area of tissue damage, whether it be permanent or reversible. When treating a tumor, the lesion includes portions of the tumor that are destroyed by the received PEF energy. When treating atrial fibrillation, the lesion includes portions of the cardiac wall where cells have been killed by the received PEF energy. Such lesions are often positioned along the entrances to the pulmonary veins in the treatment of atrial fibrillation. Such tissue modification creates a conduction block within the tissue to prevent the transmission of aberrant electrical signals.

Generally, the tissue modification systems include a specialized catheter, a high voltage biphasic waveform generator and at least one distinct energy delivery algorithm. Additional accessories and equipment may be utilized. It may be appreciated that a variety of catheter designs may be utilized, particularly specialized for specific treatments. For illustration purposes a simplified design is provided when describing the overall system. Such a simplified design provides focal therapy. However, it may be appreciated that a variety of other types of catheters and delivery electrodes may be used.

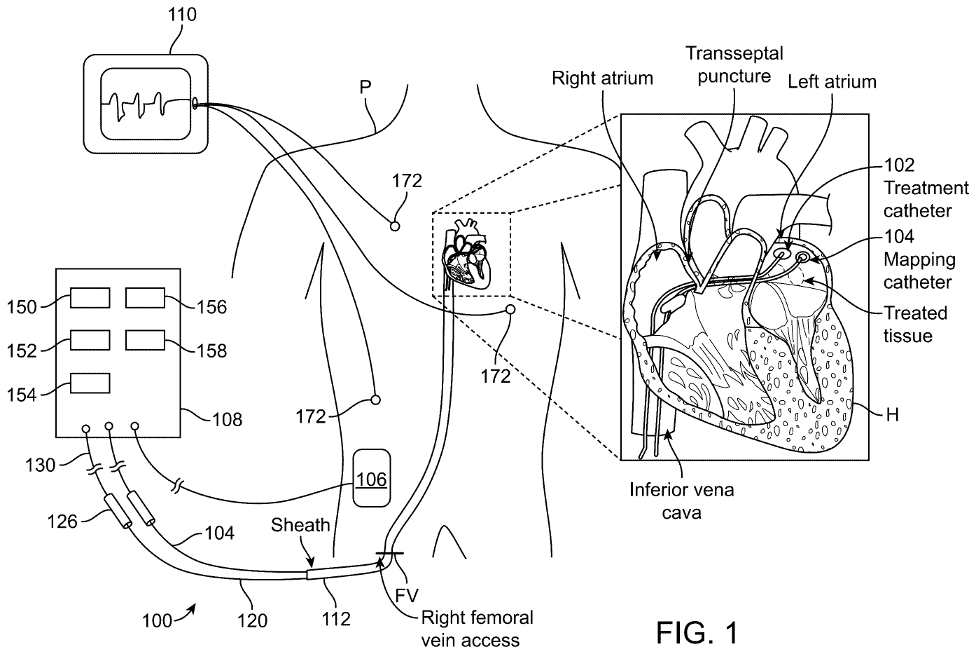

FIG. 1 illustrates an embodiment of a tissue modification system 100 used for treating cardiac tissue comprising a treatment catheter 102, a mapping catheter 104, a return electrode 106, a waveform generator 108 and an external cardiac monitor 110. It may be appreciated that although this example is specific to treating cardiac tissue, particularly for the treatment of arrhythmias, this is merely an example. Such lesion characteristics and formation may be applicable to a variety of other tissue types and locations throughout the body as mentioned herein.

In this embodiment, the heart is accessed via the right femoral vein FV by a suitable access procedure, such as the Seldinger technique. Typically, a sheath 112 is inserted into the femoral vein FV which acts as a conduit through which various catheters and/or tools may be advanced, including the treatment catheter 102 and mapping catheter 104. It may be appreciated that in some embodiments, the treatment catheter 102 and mapping catheter 104 are combined into a single device. As illustrated in FIG. 1, the distal ends of the catheters 102, 104 are advanced through the inferior vena cava, through the right atrium, through a transseptal puncture and into the left atrium so as to access the entrances to the pulmonary veins. The mapping catheter 104 is used to perform cardiac mapping which refers to the process of identifying the temporal and spatial distributions of myocardial electrical potentials during a particular heart rhythm. Cardiac mapping during an aberrant heart rhythm aims at elucidation of the mechanisms of the heart rhythm, description of the propagation of activation from its initiation to its completion within a region of interest, and identification of the site of origin or a critical site of conduction to serve as a target for treatment. Once the desired treatment locations are identified, the treatment catheter 102 is utilized to deliver the treatment energy.

In this embodiment, the proximal end of the treatment catheter 102 is electrically connected with the waveform generator 108, wherein the generator 108 is software-controlled with regulated energy output that creates high frequency short duration energy delivered to the catheter 102. It may be appreciated that in various embodiments the output is controlled or modified to achieve a desired voltage. current, or combination thereof. In this embodiment, the proximal end of the mapping catheter 104 is also electrically connected with the waveform generator 108 and the electronics to perform the mapping procedure are included in the generator 108. However, it may be appreciated that the mapping catheter 104 may alternatively be connected with a separate external device having the capability of providing the mapping procedure, such as electroanatomic mapping (EAM) systems (e.g. CARTO® systems by Biosense Webster/Johnson & Johnson, EnSite™ systems by St. Jude Medical/Abbott. KODEX-EPD system by Philips. Rhythmia HDX™ system by Boston Scientific). Likewise, in some embodiments, a separate mapping catheter 104 is not used and the mapping features are built into the catheter 102.

In this embodiment, the generator 108 is connected with an external cardiac monitor 110 to allow coordinated delivery of energy with the cardiac signal sensed from the patient P. The generator synchronizes the energy output to the patient's cardiac rhythm. The cardiac monitor provides a trigger signal to the generator 108 when it detects the patient's cardiac cycle R-wave. This trigger signal, and the generator's algorithm, reliably synchronize the energy delivery with the patient's cardiac cycle to decrease the potential for arrhythmia due to energy delivery. Typically, a footswitch allows the user to initiate and control the delivery of the energy output. The generator user interface (UI) provides both audio and visual information to the user regarding energy delivery and the generator operating status.

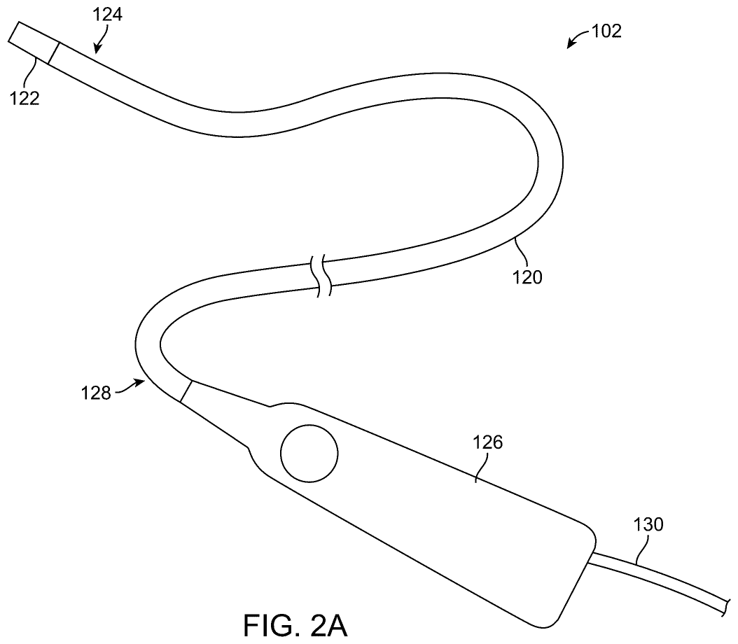

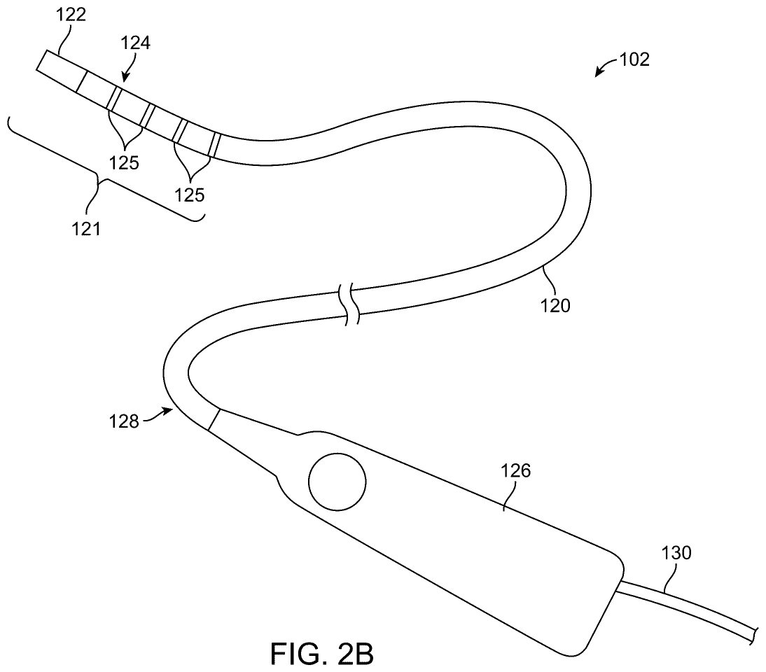

In this embodiment, the treatment catheter 102 is designed to be monopolar, wherein the distal end of the catheter 108 has as a delivery electrode 122 and the return electrode 106 is positioned upon the skin outside the body, typically on the thigh, lower back or back. FIG. 2A illustrates an embodiment of a treatment catheter 102 configured to deliver focal therapy. In this embodiment, the catheter 102 comprises an elongate shaft 120 having a delivery electrode 122 near its distal end 124 and a handle 126 near its proximal end 128. The delivery electrode 122 is shown as a “solid tip” electrode having a cylindrical shape with a distal face having a continuous surface. In some embodiments, the cylindrical shape has a diameter across its distal face of approximately 2-3 mm and a length along the shaft 120 of approximately 1 mm, 2 mm, 1-2 mm, 3 mm, 4 mm, 3-4 mm, 5 mm, 6 mm, 7 mm, 8 mm, 9 mm, 10 mm, etc. It may be appreciated that such electrodes are typically hollow yet are referred to as solid due to visual appearance. In some embodiments, the catheter 102 has an overall length of 50-150 cm, preferably 100-125 cm, more preferably 110-115 cm. Likewise, in some embodiments, it has a 7 Fr outer diameter 3-15 Fr, preferably 4-12 Fr, more preferably 7-8.5 Fr. It may be appreciated that in some embodiments, the shaft 120 has a deflectable end portion 121 and optionally the deflectable end portion 121 may have a length of 50-105 mm resulting in curves with diameters ranging from approximately 15 to 55 mm. Deflection may be achieved by a variety of mechanism including a pull-wire which extends to the handle 126. Thus, the handle 126 is used to manipulate the catheter 102, particularly to steer the distal end 124 during delivery and treatment. Energy is provided to the catheter 102, and therefore to the delivery electrode 122, via a cable 130 that is connectable to the generator 108.

Pulsed electric fields (PEFs) are provided by the generator 108 and delivered to the tissue through the delivery electrode 122 placed on or near the targeted tissue area. It may be appreciated that in some embodiments, the delivery electrode 122 is positioned in contact with a conductive substance which is likewise in contact with the targeted tissue. Such solutions may include isotonic or hypertonic solutions. These solutions may further include adjuvant materials, such as chemotherapy or calcium, to further enhance the treatment effectiveness both for the focal treatment as well as potential regional infiltration regions of the targeted tissue types. High voltage, short duration biphasic electric pulses are then delivered through the electrode 122 in the vicinity of the target tissue. These electric pulses are provided by at least one energy delivery algorithm 152. In some embodiments, each energy delivery algorithm 152 prescribes a signal having a waveform comprising a series of energy packets wherein each energy packet comprises a series of high voltage pulses. In such embodiments, the algorithm 152 specifies parameters of the signal such as energy amplitude (e.g., voltage) and duration of applied energy, which is comprised of the number of packets, number of pulses within a packet, and the fundamental frequency of the pulse sequence, to name a few. Additional parameters may include switch time between polarities in biphasic pulses, dead time between biphasic cycles, and rest time between packets, which will be described in more detail in later sections. There may be a fixed rest period between packets, or packets may be gated to the cardiac cycle and are thus variable with the patient's heart rate. There may be a deliberate, varying rest period algorithm or no rest period may also be applied between packets. A feedback loop based on sensor information and an auto-shutoff specification, and/or the like, may be included.

It may be appreciated that in various embodiments the treatment catheter 102 includes a variety of specialized features. For example, in some embodiments, the catheter 102 includes a mechanism for real-time measurement of the contact force applied by the catheter tip to a patient's heart wall during a procedure. In some embodiments, this mechanism is included in the shaft 120 and comprises a tri-axial optical force sensor which utilizes white light interferometry. By monitoring and modifying the applied force throughout the procedure, the user is able to better control the catheter 102 so as to create more consistent and effective lesions.

In some embodiments, the catheter 102 includes one or more additional electrodes 125 (e.g. ring electrodes) positioned along the shaft 120, such as illustrated in FIG. 2B, proximal to the delivery electrode 122. In some embodiments, some or all of the additional electrodes can be used for stimulating and recording (for electrophysiological mapping), so a separate cardiac mapping catheter is not needed when using catheter 102 for lesion creation, or for other purposes such as sensing, etc.

In some embodiments, the catheter 102 includes a thermocouple temperature sensor. optionally embedded in the delivery electrode 122. Likewise, in some embodiments the catheter 102 includes a lumen which may be used for irrigation and/or suction. Typically, the lumen connects with one or more ports along the distal end of the catheter 102, such as for the injection of isotonic saline solution to irrigate or for the removal of, for example, microbubbles.

In some embodiments, the catheter 102 includes one or more sensors that can be used to determine temperature, impedance, resistance, capacitance, conductivity, permittivity, and/or conductance, to name a few. In some embodiments, one or more of the electrodes act as the one or more sensors. In other embodiments, the one or more sensors are separate from the electrodes. Sensor data can be used to plan the therapy, monitor the therapy and/or provide direct feedback via the processor 154. which can then alter the energy-delivery algorithm 152. For example, impedance measurements can be used to determine not only the initial dose to be applied but can also be used to determine the need for further treatment, or not.

Referring back to FIG. 1, in this embodiment the generator 108 includes a user interface 150. one or more energy delivery algorithms 152, a processor 154, a data storage/retrieval unit 156 (such as a memory and/or database), and an energy-storage sub-system 158 which generates and stores the energy to be delivered. In some embodiments, one or more capacitors are used for energy storage/delivery. however any other suitable energy storage element may be used. In addition, one or more communication ports are included.

In some embodiments, the generator 108 includes three sub-systems: 1) a high-energy storage system, 2) a high-voltage, medium-frequency switching amplifier, and 3) the system controller, firmware, and user interface. In this embodiment, the system controller includes a cardiac synchronization trigger monitor that allows for synchronizing the pulsed energy output to the patient's cardiac rhythm. The generator takes in alternating current (AC) mains to power multiple direct current (DC) power supplies. The generator's controller can cause the DC power supplies to charge a high-energy capacitor storage bank before energy delivery is initiated. At the initiation of therapeutic energy delivery, the generator's controller, high-energy storage banks and a bi-phasic pulse amplifier can operate simultaneously to create a high-voltage, medium frequency output.

It will be appreciated that a multitude of generator electrical architectures may be employed to execute the energy delivery algorithms. In particular, in some embodiments, advanced switching systems are used which are capable of directing the pulsed electric field circuit to the energy delivering electrodes separately from the same energy storage and high voltage delivery system. Further, generators employed in advanced energy delivery algorithms employing rapidly varying pulse parameters (e.g., voltage, frequency, etc.) or multiple energy delivery electrodes may utilize modular energy storage and/or high voltage systems, facilitating highly customizable waveform and geographical pulse delivery paradigms. It should further be appreciated that the electrical architecture described herein above is for example only. and systems delivering pulsed electric fields may or may not include additional switching amplifier components.

The user interface 150 can include a touch screen and/or more traditional buttons to allow for the operator to enter patient data, select a treatment algorithm (e.g., energy delivery algorithm 152), initiate energy delivery, view records stored on the storage/retrieval unit 156, and/or otherwise communicate with the generator 108.

In some embodiments, the user interface 150 is configured to receive operator-defined inputs. The operator-defined inputs can include a duration of energy delivery or dose time, one or more other timing aspects of the energy delivery pulse, power, and/or mode of operation, one or more desired lesion dimensions, type of treatment, treatment location, type of tissue, type of catheter or electrode configuration, temperature limitations, muscle contraction limitations, and a combination thereof, to name a few.

Example modes of operation can include (but are not limited to): system initiation and self-test, operator input, algorithm selection, pre-treatment system status and feedback, energy delivery, post energy delivery display or feedback, treatment data review and/or download, software update, or any combination or subcombination thereof.

As mentioned, in some embodiments the system 100 also includes a mechanism for acquiring an electrocardiogram (ECG), such as an external cardiac monitor 110, in situations wherein cardiac synchronization is desired. Example cardiac monitors are available from AccuSync Medical Research Corporation and Ivy Biomedical Systems, Inc. In some embodiments, the external cardiac monitor 110 is operatively connected to the generator 108. The cardiac monitor 110 can be used to continuously acquire an ECG signal. External electrodes 172 may be applied to the patient P to acquire the ECG. The generator 108 analyzes one or more cardiac cycles and identifies the beginning of a time period during which it is safe to apply energy to the patient P, thus providing the ability to synchronize energy delivery with the cardiac cycle. In some embodiments, this time period is within milliseconds of the R wave (of the ECG QRS complex) to avoid induction of an arrhythmia, which could occur if the energy pulse is delivered on a T wave. It will be appreciated that such cardiac synchronization is typically utilized when using monopolar energy delivery, however it may be utilized as part of other energy delivery methods.

In some embodiments, the processor 154, among other activities, modifies and/or switches between the energy-delivery algorithms, monitors the energy delivery and any sensor data, and reacts to monitored data via a feedback loop. In some embodiments, the processor 154 is configured to execute one or more algorithms for running a feedback control loop based on one or more measured system parameters (e.g., current), one or more measured tissue parameters (e.g., impedance), and/or a combination thereof.

The data storage/retrieval unit 156 stores data, such as related to the treatments delivered, and can optionally be downloaded by connecting a device (e.g., a laptop or thumb drive) to a communication port. In some embodiments, the device has local software used to direct the download of information. such as, for example, instructions stored on the data storage/retrieval unit 156 and executable by the processor 154. In some embodiments, the user interface 150 allows for the operator to select to download data to a device and/or system such as, but not limited to, a computer device, a tablet, a mobile device, a server, a workstation, a cloud computing apparatus/system, and/or the like. The communication ports, which can permit wired and/or wireless connectivity, can allow for data download, as just described but also for data upload such as uploading a custom algorithm or providing a software update.

As described herein, a variety of energy delivery algorithms 152 are programmable, or can be pre-programmed, into the generator 108, such as stored in memory or data storage/retrieval unit 156. Alternatively, energy delivery algorithms can be added into the data storage/retrieval unit to be executed by processor 154. Each of these algorithms 152 may be executed by the processor 154.

It may be appreciated that in some embodiments the system 100 includes an automated treatment delivery algorithm that dynamically responds and adjusts and/or terminates treatment in response to inputs such as temperature, impedance at various voltages or AC frequencies, treatment duration or other timing aspects of the energy delivery pulse, treatment power and/or system status.

As mentioned, in some embodiments, the cardiac monitor provides a trigger signal to the generator 108 when it detects the patient's cardiac cycle R-wave. This trigger signal, and the generator's algorithm, reliably synchronize the energy delivery with the patient's cardiac cycle to decrease the potential for arrhythmia due to energy delivery. This trigger is within milliseconds of the peak of the R wave (of the ECG QRS complex) to avoid induction of an arrhythmia, which could occur if the energy pulse is delivered on a T wave, and also to ensure that energy delivery occurs at a consistent phase of cardiac contraction. It will be appreciated that such cardiac synchronization is typically utilized when using monopolar energy delivery, however it may be utilized as part of other energy delivery methods.

As mentioned previously, one or more energy delivery algorithms 152 are programmable, or can be pre-programmed, into the generator 108 for delivery to the patient P. The one or more energy delivery algorithms 152 specify electric signals which provide energy delivered to the walls of the heart which are non-thermal (e.g. below a threshold for thermal ablation; below a threshold for inducing coagulative thermal damage), reducing or avoiding inflammation, and/or preventing denaturation of stromal proteins in the luminal structures. In general, the algorithm 152 is tailored to affect tissue to a pre-determined depth and/or volume and/or to target specific types of cellular responses to the energy delivered.

FIG. 3 illustrates an embodiment of a waveform 400 of a signal prescribed by an energy delivery algorithm 152. Here, two packets are shown, a first packet 402 and a second packet 404, wherein the packets 402. 404 are separated by a rest period 406. In this embodiment, each packet 402, 404 is comprised of a first biphasic cycle (comprising a first positive pulse peak 408 and a first negative pulse peak 410) and a second biphasic cycle (comprising a second positive pulse peak 408′ and a second negative pulse peak 410′). The first and second biphasic pulses are separated by dead time 412 (i.e. a pause) between each biphasic cycle. In this embodiment, the biphasic pulses are symmetric so that the set voltage 416 is the same for the positive and negative peaks. Here, the biphasic, symmetric waves are also square waves such that the magnitude and time of the positive voltage wave is approximately equal to the magnitude and time of the negative voltage wave.

### A. Voltage

The voltages used and considered may be the tops of square-waveforms, may be the peaks in sinusoidal or sawtooth waveforms, or may be the RMS voltage of sinusoidal or sawtooth waveforms. In some embodiments, the energy is delivered in a monopolar fashion and each high voltage pulse or the set voltage 416 is between about 500 V to 10,000 V, particularly about 3500 V to 4000 V, about 3500 V to 5000 V, about 3500 V to 6000 V, including all values and subranges in between including about 250 V, 500 V, 1000 V, 1500 V, 2000V, 2500 V, 3000 V, 3500 V, 4000 V, 4500 V, 5000 V, 5500 V, 6000 V to name a few. Voltages delivered to the tissue may be based on the setpoint on the generator 104 while either taking in to account the electrical losses along the length of the device 102 due to inherent impedance of the device 102 or not taking in to account the losses along the length, i.e., delivered voltages can be measured at the generator or at the tip of the instrument.

It may be appreciated that the set voltage 416 may vary depending on whether the energy is delivered in a monopolar or bipolar fashion. In bipolar delivery, a lower voltage may be used due to the smaller, more directed electric field. The bipolar voltage selected for use in therapy is dependent on the separation distance of the electrodes, whereas the monopolar electrode configurations that use one or more distant dispersive pad electrodes may be delivered with less consideration for exact placement of the catheter electrode and dispersive electrode placed on the body. In monopolar electrode embodiments. larger voltages are typically used due to the dispersive behavior of the delivered energy through the body to reach the dispersive electrode, on the order of 10 cm to 100 cm effective separation distance. Conversely, in bipolar electrode configurations, the relatively close active regions of the electrodes, on the order of 0.5 mm to 10 cm, including 1 mm to 1 cm, results in a greater influence on electrical energy concentration and effective dose delivered to the tissue from the separation distance. For instance, if the targeted voltage-to-distance ratio is 3000 V/cm to evoke the desired clinical effect at the appropriate tissue depth (1.3 mm), if the separation distance is changed from 1 mm to 1.2 mm, this would result in a necessary increase in treatment voltage from 300 to about 360 V, a change of 20%.

### B. Frequency

It may be appreciated that the number of biphasic cycles per second of time is the frequency when a signal is continuous. In some embodiments, biphasic pulses are utilized to reduce undesired muscle stimulation, particularly cardiac muscle stimulation. In other embodiments, the pulse waveform is monophasic and there is no clear inherent frequency. Instead, a fundamental frequency may be considered by doubling the monophasic pulse length to derive the frequency. In some embodiments, the signal has a frequency in the range 50 KHz-1 MHz, more particularly 50 kHz-1000 kHz. It may be appreciated that at some voltages, frequencies at or below 100-250 kHz may cause undesired muscle stimulation. Therefore, in some embodiments, the signal has a frequency in the range of 300-800 KHz, 400-800 kHz or 500-800 KHz, such as 300 kHz, 400 KHz, 450 kHz, 500 kHz, 550 kHz, 600 kHz, 650 KHz, 700 kHz, 750 kHz, 800 KHz. In addition, cardiac synchronization is typically utilized to reduce or avoid undesired cardiac muscle stimulation during sensitive rhythm periods. It may be appreciated that even higher frequencies may be used with components which minimize signal artifacts.

### C. Voltage-Frequency Balancing

The frequency of the waveform delivered may vary relative to the treatment voltage in synchrony to retain adequate treatment effect. Such synergistic changes would include the decrease in frequency, which evokes a stronger effect, combined with a decrease in voltage, which evokes a weaker effect. For instance, in some cases the treatment may be delivered using 3000 V in a monopolar fashion with a waveform frequency of 600 kHz, while in other cases the treatment may be delivered using 2000 V with a waveform frequency of 400 KHz.

### D. Packets

As mentioned, the algorithm 152 typically prescribes a signal having a waveform comprising a series of energy packets wherein each energy packet comprises a series of high voltage pulses. The cycle count 420 is half the number of pulses within each biphasic packet. Referring to FIG. 3, the first packet 402 has a cycle count 420 of two (i.e. four biphasic pulses). In some embodiments, the cycle count 420 is set between 2 and 1000 per packet, including all values and subranges in between. In some embodiments, the cycle count 420 is 5-1000 per packet, 2-10 per packet, 2-20 per packet, 2-25 per packet, 10-20 per packet, 20 per packet, 20-30 per packet, 25 per packet, 20-40 per packet, 30 per packet, 20-50 per packet, 30-60 per packet, up to 60 per packet, up to 80 per packet, up to 100 per packet, up to 1,000 per packet or up to 2,000 per packet, including all values and subranges in between.

The packet duration is determined by the cycle count, among other factors. For a matching pulse duration (or sequence of positive and negative pulse durations for biphasic waveforms), the higher the cycle count, the longer the packet duration and the larger the quantity of energy delivered. In some embodiments, packet durations are in the range of approximately 50 to 1000 microseconds, such as 50 μs, 60 μs, 70 μs, 80 μs, 90 μs, 100 μs, 125 μs, 150 μs, 175 μs, 200 μs, 250 μs, 100 to 250 μs, 150 to 250 μs, 200 to 250 μs, 500 to 1000 μs to name a few. In other embodiments, the packet durations are in the range of approximately 100 to 1000 microseconds, such as 150 μs, 200 μs, 250 μs, 500 μs, or 1000 μs.

The number of packets delivered during treatment, or packet count, typically includes 1 to 250packets including all values and subranges in between. In some embodiments, the number of packets delivered during treatment comprises 10 packets, 15 packets, 20 packets, 25 packets, 30 packets or greater than 30 packets.

### E. Rest Period

In some embodiments, the time between packets, referred to as the rest period 406, is set between about 0.001 seconds and about 5 seconds, including all values and subranges in between. In other embodiments, the rest period 406 ranges from about 0.01-0.1 seconds, including all values and subranges in between. In some embodiments, the rest period 406 is approximately 0.5 ms-500 ms, 1-250 ms, or 10-100 ms to name a few.

### F. Batches

In some embodiments, the signal is synced with the cardiac rhythm so that each packet is delivered synchronously within a designated period relative to the heartbeats, thus the rest periods coincide with the heartbeats. It may be appreciated that the packets that are delivered within each designated period relative to the heartbeats may be considered a batch or bundle. Thus, each batch has a desired number of packets so that at the end of a treatment period, the total desired number of packets have been delivered. Each batch may have the same number of packets, however in some embodiments. batches have varying numbers of packets.

In some embodiments, only one packet is delivered between heartbeats. In such instances, the rest period may be considered the same as the period between batches. However, when more than one packet is delivered between batches, the rest time is typically different than the period between batches. In such instances, the rest time is typically much smaller than the period between batches. In some embodiments, each batch includes 1-10 packets, 1-5 packets, 1-4 packets, 1-3 packets, 2-3 packets, 2packets, 3 packets 4 packets 5 packets, 5-10 packets, to name a few. In some embodiments, each batch has a period of 0.5 ms-1 sec, 1 ms-1 sec, 10 ms-1 sec, 10 ms-100 ms, to name a few. In some embodiments, the period between batches is variable, depending on the heart rate of the patient. In some instances, the period between batches is 0.25-5 seconds.

Treatment of a tissue area ensues until a desired number of batches are delivered to the tissue area. In some embodiments, 2-50 batches are delivered per treatment, wherein a treatment is considered treatment of a particular tissue area. In other embodiments, treatments include 5-40 batches, 5-30batches, 5-20 batches, 5-10 batches, 5 batches, 6 batches, 7 batches, 8 batches, 9 batches, 10 batches, 10-15 batches, etc.

### G. Switch Time and Dead Time

A switch time, also known as inter-phase delay, is a delay or period of no energy that is delivered between the positive and negative peaks of a biphasic pulse, as illustrated in FIG. 3. In some embodiments, the switch time ranges between about 0 to about 1 microsecond, including all values and subranges in between. In other embodiments, the switch time ranges between 1 and 20 microseconds, including all values and subranges in between. In other embodiments, the switch time ranges between about 2 to about 8 microsecond, including all values and subranges in between.

Delays may also be interjected between each biphasic cycle, referred as “dead-time” or inter-pulse delay. Dead time occurs within a packet, but between biphasic pulses. This is in contrast to rest periods or inter-packet delays which occur between packets. In other embodiments, the dead time 412 is in a range of approximately 0 to 0.5 microseconds, 0 to 10 microseconds, 2 to 5 microseconds, 0 to 20 microseconds, about 0 to about 100 microseconds, or about 0 to about 100 milliseconds, including all values and subranges in between. In some embodiments, the dead time 412 is in the range of 0.2 to 0.3 microseconds. Dead time may also be used to define a period between separate, monophasic, pulses within a packet.

Delays, such as switch times and dead times, are introduced to a packet to reduce the effects of biphasic cancellation within the waveform. In some instances, the switch time and dead time are both increased together to strengthen the effect. In other instances, only switch time or only dead time are increased to induce this effect.

### G. Waveforms

FIG. 3 illustrated an embodiment of a waveform 400 having symmetric pulses such that the voltage and duration of pulse in one direction (i.e., positive or negative) is equal to the voltage and duration of pulse in the other direction. It may be appreciated that in other embodiments the waveform 400 has voltage imbalance. For example, each packet 402, 404 may be comprised of a first biphasic cycle (comprising a first positive pulse peak 408 having a first voltage VI and a first negative pulse peak 410 having a second voltage V2) and a second biphasic cycle (comprising a second positive pulse peak 408′ having first voltage V1 and a second negative pulse peak 410′ having a second voltage V2). Here the first voltage V1 is greater than the second voltage V2. The first and second biphasic cycles are separated by dead time 412 between each pulse. Thus, the voltage in one direction (i.e., positive or negative) is greater than the voltage in the other direction so that the area under the positive portion of the curve does not equal the area under the negative portion of the curve. This unbalanced waveform may result in a more pronounced treatment effect. It may be appreciated that in some embodiments, imbalance includes pulses having pulse widths of unequal duration. In some embodiments, the biphasic waveform is unbalanced, such that the voltage in one direction is equal to the voltage in the other direction, but the duration of one direction (i.e., positive or negative) is greater than the duration of the other direction, so that the area under the curve of the positive portion of the waveform does not equal the area under the negative portion of the waveform.

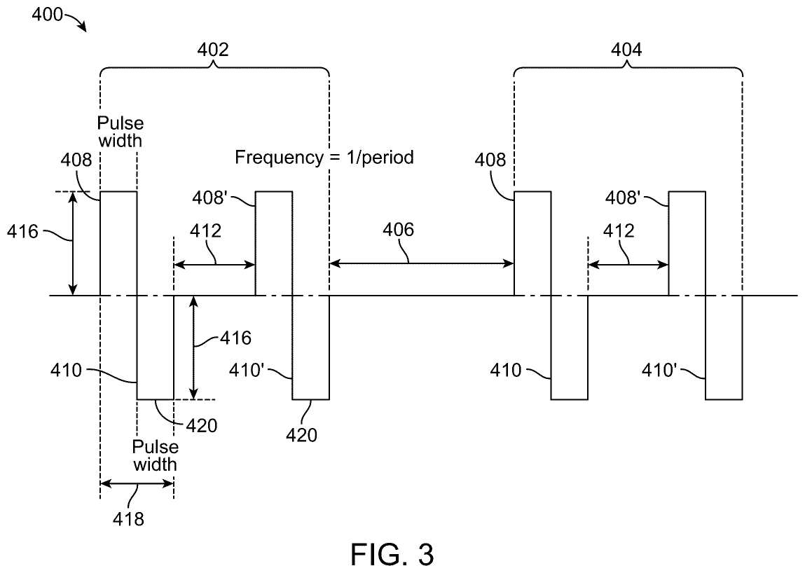

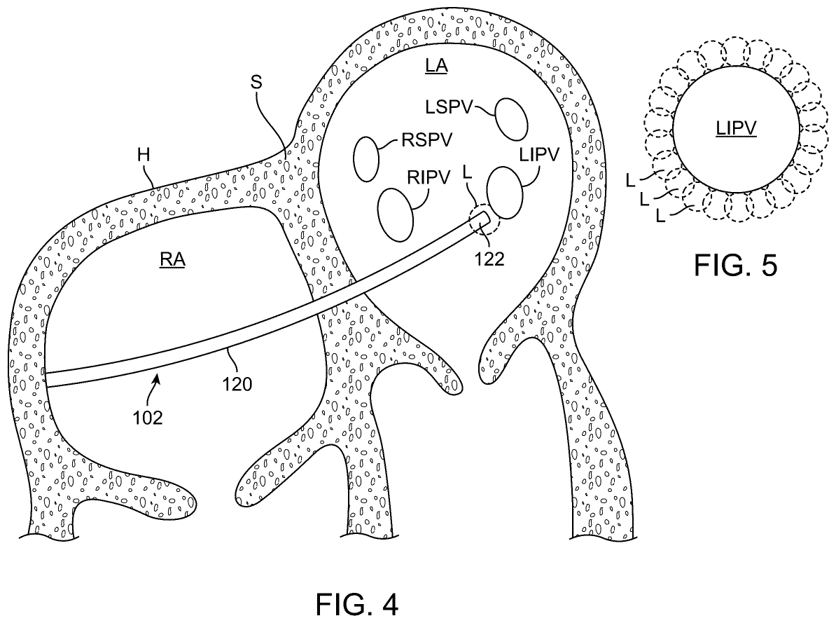

FIG. 4 illustrates a portion of the heart H showing a cut-away of the right atrium RA and left atrium LA. The largest pulmonary veins are the four main pulmonary veins (right superior pulmonary vein RSPV, right inferior pulmonary vein RIPV, left superior pulmonary vein LSPV and left inferior pulmonary vein LIPV), two from each lung that drain into the left atrium LA of the heart H. Each pulmonary vein is linked to a network of capillaries in the alveoli of each lung and bring oxygenated blood to the left atrium LA. The left atrial musculature extends from the left atrium LA and envelopes the proximal pulmonary veins. The superior veins, which have longer muscular sleeves, have been reported to be more arrhythmogenic than the inferior veins. In general, the length of the pulmonary vein sleeves varies between 13 mm and 25 mm. Pulmonary vein morphology has been reported to influence arrhythmogenesis. Likewise, cellular electrophysiology and other aspects of the pulmonary veins are associated with arrhythmogenesis and propagation.

A variety of methods are used to determine which tissue is targeted for treatment, such as anatomical indications and cardiac mapping. Typically, a mapping catheter is chosen to desirably fit the pulmonary vein, adapting to the size and anatomical form of the pulmonary vein. The mapping catheter allows recording of the electrograms from the ostium of the pulmonary vein and from deep within the pulmonary vein; these electrograms are displayed and timed for the user. The treatment catheter 102 is initially placed deep within the pulmonary vein and gradually withdrawn to the ostium, proximal to the mapping catheter. Mapping and treatment then commences.

The current understanding of pulmonary vein electrophysiology is that most of the fibers in the pulmonary vein are circular and do not carry conduction into the vein. The electrical conduction pathways are longitudinal fibers which extend between the left atrium LA and the pulmonary vein. Pulmonary vein isolation is achieved by ablation of these connecting longitudinal fibers. For the left-sided pulmonary veins, pacing of the distal coronary sinus tends to increase the separation of the atrial signal and the pulmonary vein potential making these more electrically visible. The signals from within the pulmonary vein are evaluated. Each individual signal consists of a far field atrial signal, which is generally of low amplitude, and a sharp local pulmonary vein spike. The earliest pulmonary vein spike represents the site of the connection of the pulmonary vein and atrium. If the pulmonary vein spike and the atrial potential are examined, on some of the poles of the mapping catheter, these electrograms are widely separated, at other sites there will be a fusion potential of the atrial and PV signal. The latter indicate the sites of the longitudinal fibers and the potential sites for treatment.

In some embodiments, the tissue surrounding the opening of the left inferior pulmonary vein LIPV is treated in a point by point fashion with the use of the treatment catheter 102 (with assistance of mapping) to create a circular treatment zone around the left inferior pulmonary vein LIPV, as illustrated in FIG. 5. In some instances, specialized navigation software can be used to allow appropriate positioning of the treatment catheter 120. The delivery electrode 122 is positioned near or against the target tissue area, and energy is provided to the delivery electrode 122 so as to create a treatment area or lesion L. Since the energy is delivered to a localized area (focal delivery), the electrical energy is concentrated over a smaller surface area, resulting in stronger effects than delivery through an electrode extending circumferentially around the lumen or ostium. It also forces the electrical energy to be delivered in a staged regional approach, mitigating the potential effect of preferential current pathways through the surrounding tissue. These preferential current pathways are regions with electrical characteristics that induce locally increased electric current flow therethrough rather than through adjacent regions. Such pathways can result in an irregular electric current distribution around the circumference of a targeted lumen, which thus can distort the electric field and cause an irregular increase in treatment effect for some regions and a lower treatment effect in other regions. This may be mitigated or avoided with the use of focal therapy which stabilizes the treatment effect around the circumference of the targeted region. Thus. by providing the energy to certain regions at a time, the electrically energy is “forced” across different regions of the circumference, ensuring an improved degree of treatment circumferential regularity. FIG. 5 illustrates the repeated application of energy in point by point fashion around the left inferior pulmonary vein LIPV with the use of the treatment catheter 102 to create a circular treatment zone. As illustrated, in this embodiment each lesion L overlaps an adjacent lesion L so as to create a continuous treatment zone. It may be appreciated that the number of lesions L may vary depending on a variety of factors. particularly the unique conditions of each patient's anatomy and electrophysiology. In some embodiments, the number of lesions L include one, two, three, four, five, six, seven, eight, nine, ten, fifteen, twenty, twenty-five, thirty or more.

The size and depth of each lesion L may depend on a variety of factors, such as parameter values, treatment times, tissue characteristics, etc. It may be appreciated that the effect of the energy on the tissue is a function of both the intensity of the waveform pulses as well as the cumulative injury incurred by a progressive series of those pulses caused by the dose. When considering generating a particular lesion size, an effective electric field threshold may be defined by, for example, waveform characteristics (e.g. fundamental frequency, packet active period, total number of packets) and a given electrode type or arrangement. This can be fine-tuned with changes in delivered voltage, which affect field intensity, and/or duration of the electric field pulse, to name a few. In addition to alterations to a pulse, there is a contribution of secondary pulse parameters, including the number of pulses that are delivered and their cadence of delivery. For cycles of biphasic pulse waveforms where the electric field polarity is alternated, the addition of multiple cycles in quick succession, comprising a packet, will alter the effective electric field threshold to induce a desired outcome.

When titrating a PEF dose for cardiac ablation, it is important to consider the relative desirable and undesirable attributes associated with each treatment parameter. The basic waveform treatment parameters are briefly discussed herein. A summary table depicting the relative implications of each is provided in Table 1, below.

Voltage: Voltage directly affects the electric field intensity distribution. Increasing voltage will directly increase treatment magnitude. However, higher voltages also increase associated tissue heating, potentially resulting in undesirable thermal effects. Increased voltage may also increase the muscle contraction associated with PEFs, the volume of gaseous microemboli generated, and the likelihood of electrical arcing, potentially resulting in char and coagulum formation or barotrauma.

Waveform Shape: PEFs may use multiple waveforms to invoke their effects, however in practice they are generally sinusoidal or square waveforms. Waveforms may be fundamentally viewed as a train of pure monophasic pulses (where a sequence of short (≤10 μs) or long (>10 μs) pulses is delivered with relatively long (>1 ms) delays between them, all of the same polarity), alternating monophasic (where a sequence of long pulses is delivered with relatively long delays between them, with alternating polarity), and biphasic (where short (≤10 μs) pulses are delivered with relatively brief delays (<1 ms) between changes in polarity). Monophasic waveforms create the strongest treatment effects. However, they may also induce muscle contraction, pressure waves, and gaseous emboli formation. Biphasic waveforms can greatly reduce muscle contraction but will generate weaker treatment effects for equivalent total packet duration and applied field. However, with optimization of waveform parameters, it is possible to balance reduced muscle contraction with treatment effects, permitting the generation of clinically relevant lesions without treatment-prohibitive muscle contraction. For biphasic waveforms, the fundamental frequency affects the treatment, where higher frequencies (i.e., shorter individual pulse durations) monotonically reduce muscle contraction but also result in progressively smaller treatment effects. It should be noted that PEF waveforms are adjustable as a spectrum. Asymmetry and the fundamental frequency of the waveform may be adjusted to further titrate effects, where greater asymmetry or lower fundamental frequencies increase treatment effect and muscle contraction, until approaching purely monophasic waveforms.

Collective Pulse Duration: When multiple pulses are delivered in rapid succession (e.g., as a packet), their effects may be viewed as a collective pulse. As the packet duration increases, corresponding increases in treatment effect, muscle contraction, gas formation, heating, and these effects can increase the chance for electrical arcing.

Total Activation Duration: When multiple packets are delivered to tissue, the accumulated injury increases. Thus, more packets will result in stronger treatment effects, but will increase the treatment delivery time (for a matched packet delivery rate) and will increase the cumulative temperature rise if there is insufficient time for heat dissipation, such as between subsequent packets.

Packet Delivery Rate: When a pause is provided between a sequence of pulses (e.g. a packet), the tissue has an opportunity to conduct heat away from the warmed tissues, decreasing total temperature rise. However, during this pause, cells continue to expend energy in an effort to recover from PEF effects. When a sequence of pulses is delivered with a relatively larger pause (milliseconds to tens of seconds). cellular recovery is generally unaffected but the total tissue temperature rise is reduced. This enables delivery of strong cumulative treatments while maintaining acceptable levels of temperature rise. In some instances, delivering a series of pulses or packets spread over a longer period of time will increase treatment effectiveness.

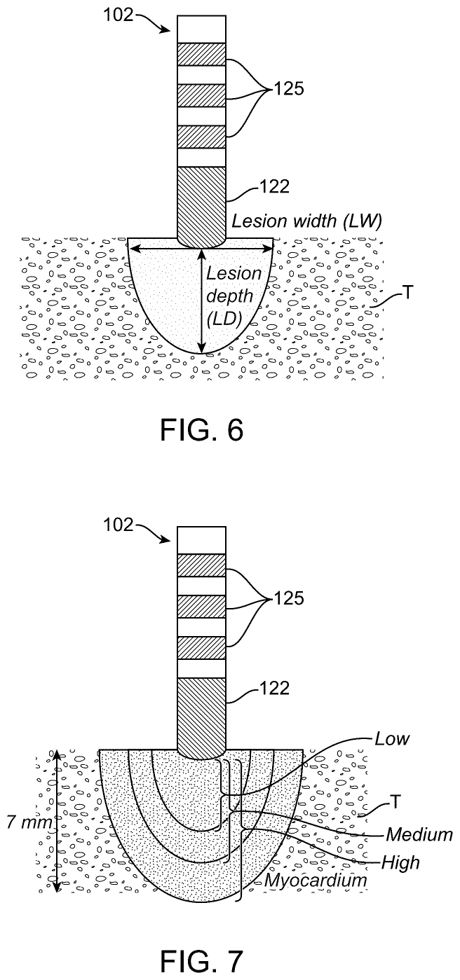

As mentioned previously, the size and depth of each lesion L may depend on a variety of factors. FIG. 6 is a schematic illustration of a lesion L generated by a catheter 102, such as illustrated in FIG. 2B. Here, the delivery electrode 122 is positioned against the tissue T, such as cardiac tissue, and energy is provided to the delivery electrode 122 so as to create a lesion L. This delivery electrode 122 creates a lesion L having a three-dimensional parabolic shape or rounded cone shape. As illustrated, the lesion L has a lesion depth LD (extending from the tip of the delivery electrode 122 to the furthest depth of the parabola shape) and a lesion width LW (extending across the base of the parabola). The lesion depth LD and lesion width LW vary depending on the delivered energy. In particular, the lesion dimensions vary depending on the parameters of the waveform and the dose, among other influences such as tissue type, etc. Correlations between lesion dimensions and changes in parameters and dose of the PEF energy described herein have been analyzed to generate predictive algorithms. Such algorithms are used in a variety of tissue modification systems 100 to improve clinical usability, treatment control and patient outcomes. This will be described in more detail in later sections.

FIG. 7 illustrates three different levels or doses of energy delivery. In this example, the “low” dose is a combination of parameters that provides the smallest lesion size (e.g. 3-4 mm width, 4-5 mm depth). In this example, the “medium” dose provides results in a mid-level sized lesion (e.g. 7-8 mm width. 5-6 mm depth). And, in this example, the “high” dose results in the largest lesion size (9-10 mm width. 7-8 mm depth). It may be appreciated that such terminology of low, medium and high are simply relative to each other for the purposes of this example. Moving from low to high, both the peak amplitude (V or I) and the number of individual packets delivered increases for a given treatment. In some embodiments, such terms correlate values as provided in Table 2, below.

TABLE 2

| | | | | | | |
|---|---|---|---|---|---|---|
| Energy Setting | 19 | Amps | 22 | Amps | 25 | Amps |
| On Time per treatment | 1.4 | ms | 2.4 | ms | 3.4 | ms |

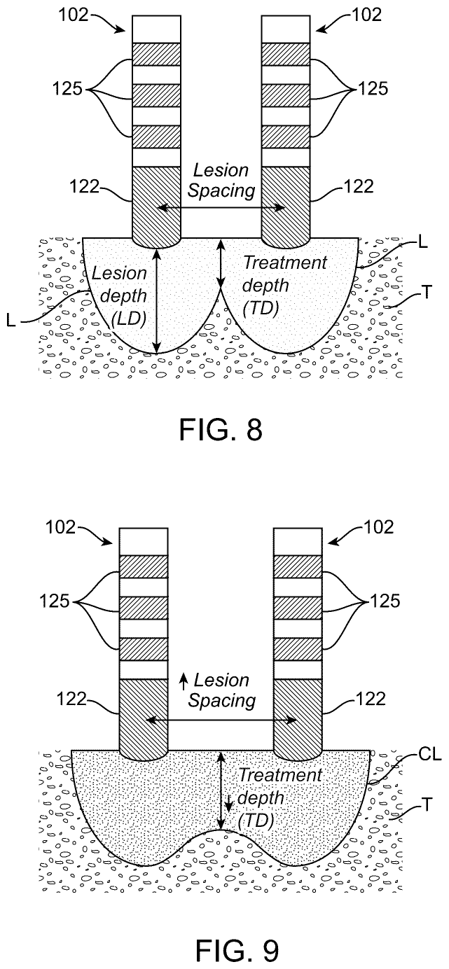

Referring back to FIG. 5, continuous lesions are formed by the repeated application of energy in point-by-point fashion, typically wherein each lesion L overlaps an adjacent lesion L. The desired spacing between the lesions L, and therefore the amount of overlap, depends at least on the size of the lesion L generated. This in turn depends on a variety of factors described herein. FIG. 8 illustrates the generation of two adjacent lesions L that are overlapping. In this embodiment, the lesions L are created in succession utilizing the same catheter 102. The actual treatment depth TD, considered the minimum depth of the formed lesion, is located in between both focal lesions L at the point of overlap. As illustrated, this is smaller than the lesion depth LD of either lesion L. FIG. 9 illustrates the resultant continuous lesion CL formed from the energy delivered by the catheter 102.

During treatment, physicians look at several cues to determine where they should treat next in a point-by-point sequence. For example, a physician may consider impedance measurements. electrogram signals, mapping system placement, fluoroscopy, etc. In the field of radiofrequency ablation, known protocols are often followed, such as the CLOSE protocol. However, such protocols are inapplicable to PEF energy delivery and would erroneously direct the physician to utilize lesion placements that would result in gaps and therefore failed conduction blocks. This is due to transient effects of energy delivery in portions of the lesion considered a “stun” effect.

When creating a lesion using PEF energy, a portion of the tissue receiving the energy will be killed by the PEF therapy, ultimately being replaced by transmural fibrotic scar. And, another portion of the tissue will be stunned by the PEF therapy, acutely experiencing a marked decrease in electrical conduction (i.e., “stunned”) but ultimately surviving the PEF therapy in at least the short-to medium-term. The stunning phenomenon is likely the result of a penumbral band of injured, but not killed cells in the region surrounding lethally affected tissue. This penumbra effect may have pronounced implications on clinical outcomes by confounding the ability to accurately determine intraprocedural treatment efficacy. To ensure durable clinical efficacy from PEF cardiac ablation procedures, it is important to ensure that the acute electrical isolation is not reliant on the stunned tissue, but instead reflects the killed tissue that will result in a durable fibrotic scar.

A variety of measures have been undertaken to determine the boundary of the stunned area and the killed area. In some instances, a swine heart model has been utilized. In one example, a 7F treatment catheter 102 with a 3.5 mm ablation electrode 122 was connected to a pulsed electric field (PEF) generator 108 and positioned in the right ventricle RV and left ventricle LV under EnSite guidance in two closed chest pigs. Biphasic PEF current was delivered between the ablation electrode and a skin patch at 13 RV sites (28 Amp, total pulse width of 1.4 ms, 4 pulses) and 19 LV sites (35 Amp, 1.6 ms, 7 pulses). Two hours after ablation, triphenyl tetrazolium chloride (TTC) was administered. Pigs were sacrificed and hearts were excised and fixed in formalin. Hearts were sectioned and stained with hematoxy lin and cosin (H&E) and Masson trichrome. Cytochrome c oxidase (COX) staining was also performed to examine mitochondrial activity to delineate stun and kill lesion boundaries.

Ablation lesions were well demarcated with TTC staining, showing a dark central zone surrounded by pale boundaries. Histology showed destruction of myocyte architecture within the pale boundaries. A hyperstained (dark red) rim beyond the pale boundaries indicated the stun zone. COX staining showed no or low mitochondrial activity within the pale boundary, consistent with an ablated region. Enhanced activity of COX staining extended to unaffected normal myocardium, consistent with the stun zone. Therefore, acute ventricular lesions produced by PEF ablation show clear demarcation by TTC staining. COX staining suggests that killed portions of the lesions are surrounded by a stun zone.

The stun effect can be harnessed in certain electrophysiological applications. For example, different types of myocardial cells may be more sensitive to stunning than others, which can help to distinguish Purkinje origins from other types of myocardial origins. The stun effect can also serve to sub-lethally test an ablation site prior to finalizing it to ensure that there will be no inadvertent damage to the conduction system (similar to so-called “cryomapping” with cryothermy).

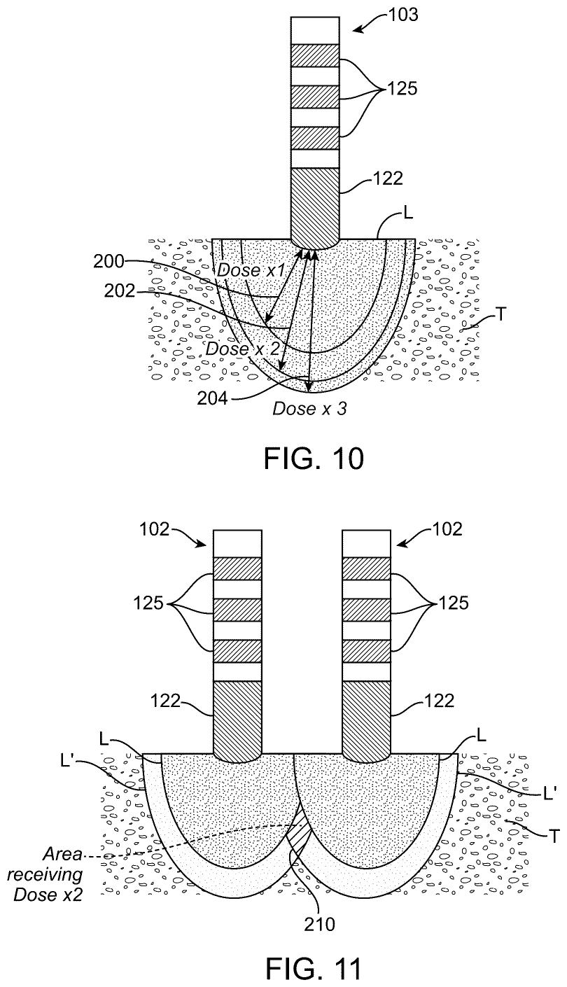

To increase the treatment depth TD, multiple doses may be utilized. FIG. 10 illustrates the effects of multiple doses on a lesion size. Here, each dose initially extends from the delivery electrode 122 toward the furthest lesion boundary. In this embodiment, the energy is delivered in a monopolar fashion and energy intensity drops off at increasing distances from the delivery electrode 122. Tissue within the outermost rings is simply “stunned” tissue while the tissue within the innermost ring receives sufficient energy to be killed. Thus, as illustrated, a single dose provides a first lesion size 200. Applying the dose again (dose x 2) treats the tissue again causing the tissue in the middle ring to tip the balance from stunned to killed. Thus, the double dose increases the first lesion size 200 to a second lesion size 202. Applying the dose again (dose×3) repeats the process and now causes the tissue in the outermost ring to tip the balance from stunned to killed as well. This increases the second lesion size 202 to a third lesion size 204. It may be appreciated that additional doses may increase the lesion size incremental amounts leading up to a limit. A numerical approximation has been employed to determine the effect of multiple delivered doses. It has been found that efficacy curves increase with packet number.

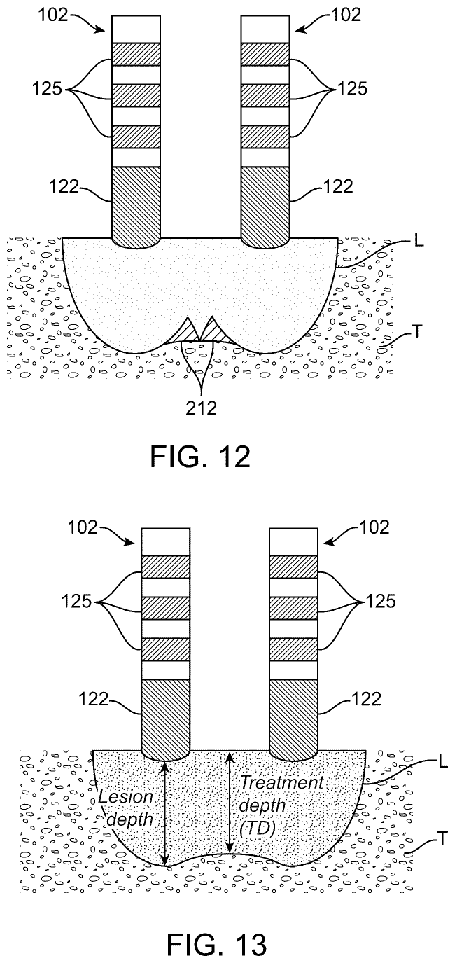

It may be appreciated that advantageously overlapping lesions also increases the treatment depth of the lesions. FIG. 11 illustrates an embodiment of two overlapping lesions L. Similar to FIG. 8. the lesions L overlap when the catheter 102 is re-positioned sufficiently close to the first placement when making the point-by-point lesions. Referring back to FIG. 11, the dose creates a larger potential lesion L′ which diminishes or reverses to a lesion the size of lesion L. However, as highlighted in FIG. 11, a zone 210 is identified that receives double dosing from both catheter 102 placements. Consequently, this zone 210 transitions from stunned to killed. This zone 210 is now considered part of the treatment depth TD of the lesion, as illustrated in FIG. 12, effectively increasing the treatment depth TD. In the similar way, some tissue 212 around the zone 210 will be affected by a fractional energy from both lesions, resulting in similar damage and can thus be considered part of the lesion. Consequently, the resulting continuous lesion L is illustrated in FIG. 13. As shown, the treatment depth TD has increased in comparison to FIG. 8.

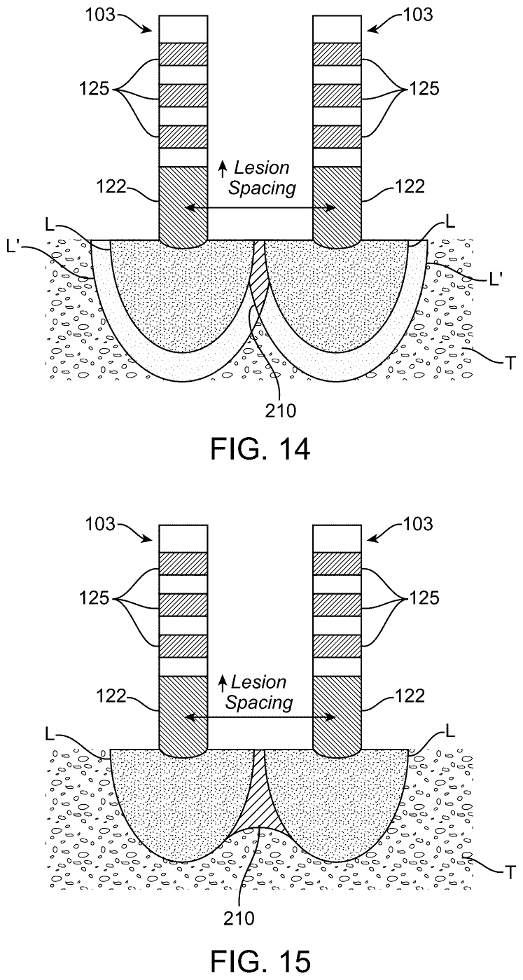

Changing the distance between the lesion locations (i.e. changing the spacing between the catheter placements) changes the amount of overlap of the lesions. Generally, increasing spacing will commonly imply a reduction of the resulting treatment depth TD. FIG. 14 illustrates lesion spacing (i.e. catheter spacing) that is larger than that of FIG. 11. Thus, the zone 210 that receives double dosing from both catheter 102 placements has shifted upwards. This in turn creates a shallower treatment depth for the continuous lesion as illustrated in FIG. 15. However, it is important that the lesion spacing is close enough that this zone 210 is created, otherwise a gap will appear between the lesions leading to failure of therapy.

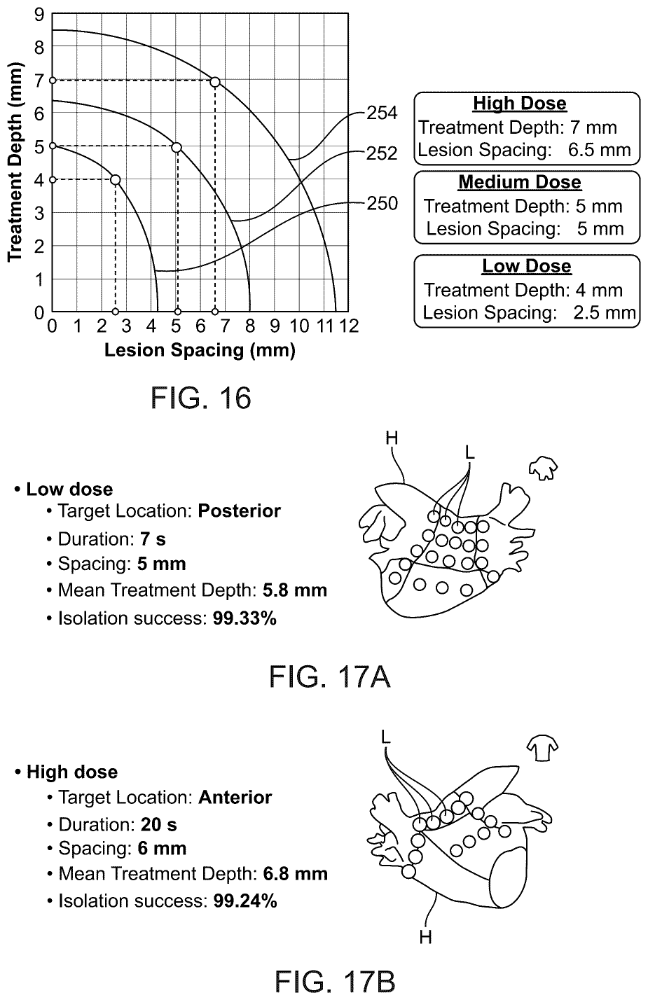

In order to estimate the expected treatment depth TD for overlapping lesions, a simplified geometrical approximation has been employed assuming ellipsoid lesions. From this the estimated treatment depth TD can be determined in relation to lesion spacing so as to generate a desired treatment protocol. FIG. 16 illustrates example correlations between treatment depth TD and lesion spacing at three different energy doses (low 250), medium 252, high 254). In some embodiments, these energy doses correlate to those of Table 2, above. To ensure a desired target treatment depth, maximum lesion spacing can be determined from the graph.

It may be appreciated that various measured atrial wall thicknesses have been reported, particularly depending on the location within the heart. Based on comparison of data, it has been determined that a 4 mm treatment depth TD is adequate for more than 99.42% of the expected wall thicknesses. Therefore, a minimum of 4 mm treatment depth TD is desired. To achieve this, the lesion spacing is determined based on the dose level. A reference dose (e.g. 22A, 400 kHz, 45 cycles, 12packets) selected as a baseline intensity for therapy with 3 mm spacing resulted in 4.0 mm treatment depth. Likewise, a dose provided with higher amperage and more packets delivered (“high” dose) (e.g. 25A, 400 KHz, 45 cycles, 21 packets) with 5 mm spacing resulted in 4.4 mm treatment depth. This spacing performed well during interventions. However, to attain 6 mm treatment depth it was found that a high dose with 3 mm spacing was utilized.

FIGS. 17A-17B illustrate the use of two different treatment protocols for different areas of the heart. FIG. 17A illustrates example lesions on a posterior surface of the heart H which has a thinner wall thickness (generally <3 mm). Here, energy was delivered through the catheter to create lesions L along the posterior surface, wherein the spacing was 5 mm and the mean treatment depth was 5.8 mm. FIG. 17B illustrates example lesions on an anterior surface of the heart H which has a thicker wall thickness (generally 2-6 mm). Here, energy was delivered through the catheter to create lesions L along the posterior surface, wherein the spacing was 6 mm and the mean treatment depth was 6 mm. The energy delivered was considered high in comparison to the energy delivered to the posterior surface because three times the number of packets were delivered. Thus, different anatomical regions may warrant different treatment doses to ensure transmural treatment effect thickness (i.e., stronger doses at regions evaluated in the patient or generally known to be thicker). Alternately, a strategy of spacing lesions closer together at different distances may be used to induce the same effect. For example, on the thin tissue, the high dose example could use spacing of 5 mm and generate contiguous transmural lesions. However, at the anterior or carinal regions, the same dose could be used with a spacing of 3 mm to also get transmural treatment effects in this thicker tissue.

It may be appreciated that the therapy concludes when all undesired electrical connections have been treated. When performing pulmonary vein isolation, the electrical connections between the atrium and the vein are treated until there is electrical silence within the pulmonary vein, with only the far field atrial signal being recorded. Occasionally spikes of electrical activity are seen within the pulmonary vein with no conduction to the rest of the atrium; these clearly demonstrate electrical discontinuity of the vein from the rest of the atrial myocardium. Additional treatment areas can be created at other locations to treat arrhythmias in either the right or left atrium dependent on the clinical presentation. Testing is then performed to ensure that each targeted pulmonary vein is effectively isolated from the body of the left atrium.

Lesions have been evaluated to ensure longevity of the results and therefore durability of the lesions. For example, a 90-day remapping study was undertaken to evaluate the results of lesions generated using three compatible focal cardiac ablation catheters. These ablation catheters were conventional catheters designed for delivery of radiofrequency energy wherein PEF energy described herein was delivered through the catheters utilizing specialized system components to enable such a procedure. The objective was to study the safety and clinical performance to evaluate catheter ablation of atrial fibrillation using the systems and methods described herein. The study design was prospective. single-arm, open-label, multi-center study. The patients were experiencing catheter ablation for the first time for treatment of paroxysmal atrial fibrillation (PAF) or persistent AF (PerAF) of short-duration (<1year). The primary safety endpoint was incidence of system and procedure-related serious adverse events of interest within 30 days of ablation. The acute secondary endpoints included pulmonary vein isolation during the index ablation procedure confirmed by both entrance and exit block. The chronic secondary endpoints included durability of pulmonary vein isolation at 90-day remapping. Additional safety assessments included peri-procedural gaseous emboli assessment on ICE and ST-segment change assessments on ECG, phrenic nerve injury, pre-and post-procedure cranial MRI for assessment of brain lesions, pre-and post-procedure stroke assessment via NIHSS, post-procedure esophageal endoscopy and PV imaging via cardiac CT/MRI.

The following three conventional RF catheters and mapping systems were used:
1) TACTICATH™ SE; ENSITE™; Advisor™ HD-Grid
2) INTELLANAV STABLEPOINT™; RHYTHMIA HDx™; INTELLAMAP ORION™
3) THERMOCOOL SMARTTOUCH™; CARTO® 3; PENTARAY®

Physicians chose 1 of 3 energy settings (low=19, medium=22, high=25Amps) to perform wide antral circumferential ablation with a target contact force of ≥5 g. Acute success was assessed via entrance and exit block. Safety was assessed by independent evaluation of pre-specified SAEs of interest within 30 days. PVI durability was assessed at 90 days with high-density mapping and confirmation of entrance and exit block. The clinical energy setting strategy was a variant of the CLOSE protocol while using PEF energy. The lesion tag diameter represented lesion spacing and varied depending on cohort. Patients were divided into five cohorts as presented in Table 3:

TABLE 3

| | | Anterior | Posterior | Lesion | |
|---|---|---|---|---|---|
| Cohort | Catheter | Setting | Setting | Spacing | |
| 1 | TACTICATH ™ SE | 22A | 19A | 5/3 | mm |
| 2 | TACTICATH ™ SE | 22A | 22A | 5 | mm |
| 3 | TACTICATH ™ SE | 25A | 22A | 4 | mm |
| 4 | INTELLANAV | 25A | 22A | 4 | mm |
| | STABLEPOINT ™ | | | | |
| 5 | THERMOCOOL | 25A | 22A | 6 | mm |
| | SMARTTOUCH ™ | | | | |

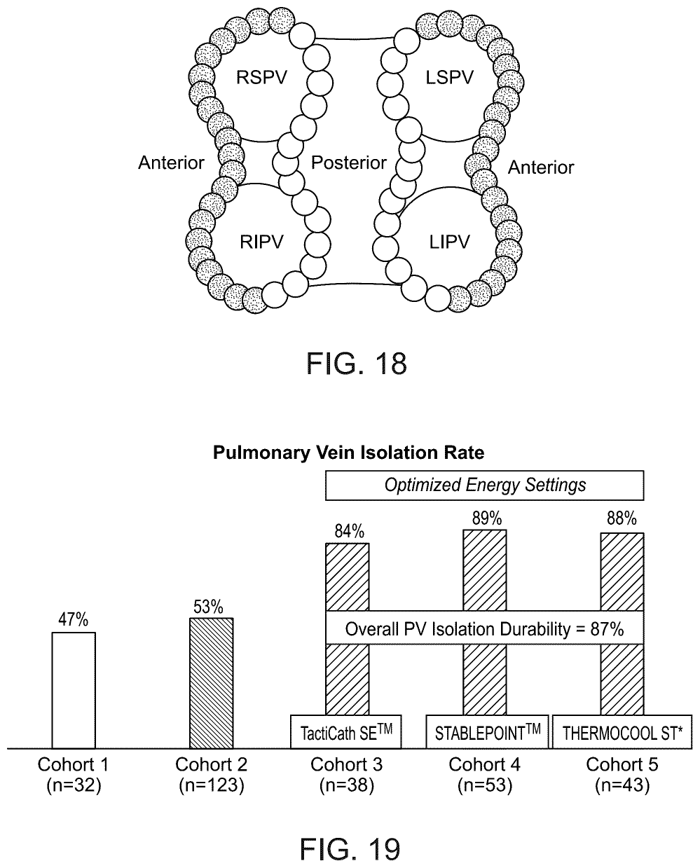

In some instances, the anterior and posterior settings differ due to thickness of the target tissue. Anterior sites tend to be thicker while posterior sites tend to be thinner. Likewise, posterior sites are typically adjacent to anatomical features that may be more sensitive to energy delivery, such as the esophagus. Example placements of anterior and posterior lesions are illustrated in FIG. 18.

Pulmonary vein isolation was achieved in all subjects, with first pass isolation in 92% of PVs (277/302). Ninety days after treatment, high-density remapping of each pulmonary vein was achieved in 74 patients with the Advisor™ HD-Grid or PENTARAY®. Circumferential mapping of the entire pulmonary vein was achieved to confirm durable, contiguous, transmural ablation. Entrance block was confirmed for each pulmonary vein. Exit block was confirmed for each pulmonary vein with high output pacing. Detailed mapping revealed focal reconnections in some pulmonary veins that initially appeared isolated. Reconnections were treated with PEF energy as described herein. The pulmonary vein isolation rates at the 90-day remap are illustrated in FIG. 19. Thus, cohort 3, 4, 5 achieved overall pulmonary vein isolation durability of 87%. This is significantly superior to those of cohort 1. 2 which ranged from 47-53%. Thus in some embodiments, a high dose (25A) with spacing of 4-6 mm for anterior locations and a medium dose (22A) with spacing of 4-6 mm for posterior locations is desired. Thus, of the 73 subjects remapped, the PV isolation rate improved from 47% to 84% as energy settings were optimized using TactiCath SE™ in Cohorts 1, 2, and 3. Applying the optimized workflow to Cohorts 4(STABLEPOINT™) and 5 (THERMOCOOL ST®), the PV isolation rates were 89% and 87%.

It was shown that effective PEF delivery was successfully integrated into standard PVI workflow with limited learning curve. No intraprocedural gaseous emboli were observed on ICE. Favorable safety profile was found for esophageal,, phrenic nerve, pulmonary veins, and brain, 100% acute isolation was achieved, but acute isolation is not a surrogate for durable isolation or freedom from atrial fibrillation. 90-day remaps were desired to optimize dose and workflow. Very high compliance for remap procedures (96%) was achieved with a comprehensive durability assessment. Improvement in durable pulmonary vein isolation rate was achieved with energy setting optimization from Cohorts 1 to 3.Maintained safety and efficacy was achieved with optimized settings across three solid tip, contact force sensing conventional RF focal catheter platforms while using PEF energy. Preliminary 12-month freedom from atrial fibrillation was achieved.

It may be appreciated that focal therapy may be delivered with a variety of catheter designs and methods. For example, in some embodiments, the energy delivery catheter 102 is configured to provide focal therapy such as according to international patent application number PCT/US2018/067504titled “OPTIMIZATION OF ENERGY DELIVERY FOR VARIOUS APPLICATIONS” which claims priority to Provisional Patent Application No. 62/610,430 filed Dec. 26, 2017 and U.S. Provisional Patent Application No. 62/693,622 filed Jul. 3, 2018, all of which are incorporated herein by reference for all purposes. Likewise, focal therapy may be delivered with a variety of catheter designs, optionally with the use of a variety of accessories, such as according to international patent application number PCT/US2020/066205 titled “TREATMENT OF CARDIAC TISSUE WITH PULSED ELECTRIC FIELDS”, filed Dec. 18, 2020 which claims priority to Provisional Patent Application No. 62/949,633 filed Dec. 18, 2019, Provisional Patent Application No. 63/000,275 filed Mar. 26, 2020 and Provisional Patent Application No. 63/083,644 filed Sep. 25, 2020, all of which are incorporated by reference for all purposes.

As mentioned previously, lesion dimensions vary depending on the parameters of the PEF waveform and the dose, among other influences such as tissue type, etc. This is the case when delivering PEF energy to cardiac tissue, as mentioned above, and to other tissue types throughout the body. Therefore, different waveforms of PEF energy and different doses of PEF energy provide different results, particularly different lesion sizes and types. Systems, devices, and methods are provided that allow a physician or user to control the PEF energy delivered to provide a desired result and/or provide predictive information with which the user can utilize to generate a desired result.

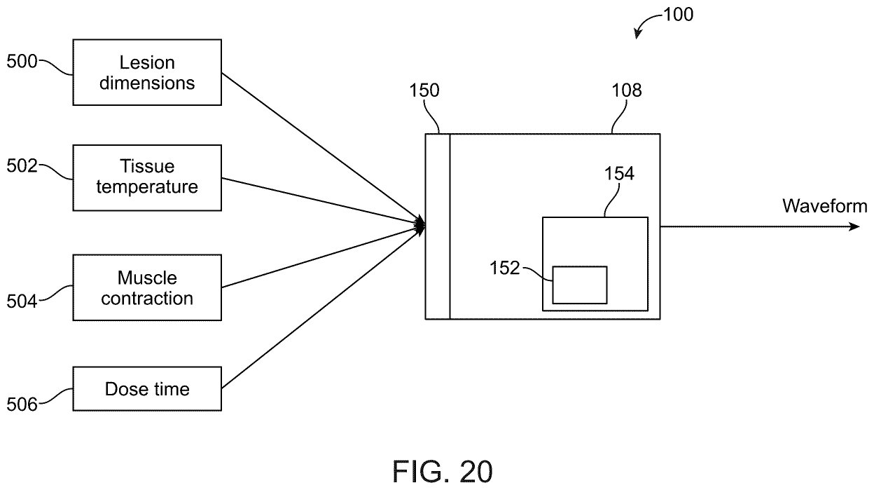

For example, in some instances, a user may enter or input treatment aspects into the system 100, such as via the user interface 150, which are used by the system 100 (e.g. generator 108) to generate or select a particular energy delivery algorithm 152 that optimizes the treatment waveform and/or dose for the desired treatment. FIG. 20 provides a schematic illustration of such a system 100. In this example, the user inputs one or more desired lesion dimensions as a first input 500. Such dimensions may include lesion depth, lesion width, lesion shape or other dimensional characteristics. The user may have such preferences based on the type of treatment (e.g. size of tumor, electrical blockade, etc.), treatment location (e.g. within a lumen, along a tissue edge, proximity to other anatomical features, etc), or type of tissue (which may also depend on treatment location), to name a few. In this example, the user inputs desired temperature characteristics, such as maximum allowable temperature rise, as a second input 502. The user may have such preferences based on type of treatment and treatment location. For example, there may be different tolerances for temperature in some types of treatment (e.g. higher temperatures may be more acceptable for tumor ablation). Likewise, some areas may be more sensitive to temperature increases or present increased risk in the presence of temperature increases (e.g. posterior wall of the left atrium adjacent to the esophagus). Thus, preferences may be based on delivery electrode proximity to sensitive structures, such as the phrenic nerve, esophagus, sinoatrial node, or atrioventricular node, where thermal damage to these structures may be irrecoverable and result in unacceptable patient morbidity or mortality. Likewise, in this example, the user inputs desired muscle contraction, such as a maximum allowable amount of muscle contraction, as a third input 504. The user may have such preferences based on delivery electrode proximity to excitable tissues of interest. For example, when the delivery electrode is near the phrenic nerve, a patient will have a much greater likelihood of diaphragmatic contraction. In some instances, the extent of this may be unacceptable at a normal dose, and thus the clinician may choose to reduce the contraction, improving catheter stability during treatment delivery. In another example, if the energy is being delivered near the vagus nerve, the contractile excitability may also be correlated to the stimulation of this nerve. Thus, by reducing the excitability of the waveform for this tissue, a user may reduce the likelihood of patient cough during a procedure, improving electroanatomical mapping accuracy. Likewise, preferences for muscle contraction may be based on the surgical management of the patient (e.g. patient being treated with conscious sedation versus general anesthesia). Or the user may have preferences based on the anatomical location and the effect that muscle contraction could have on the treatment itself (e.g., loss of tissue contact during delivery) or a safety related preference (e.g., concern for perforation when ablating on thinner tissues such as the posterior wall of the left atrium).

Further, in this example, the user inputs dose time as a fourth input 506. The dose time is the amount of time allowable for a particular application of energy. In some instances, shortened dosing times are desired due to clinical situations that restrict the allowable time to deliver energy, such as rapid contractile movement of the heart and delivering energy to difficult-to-reach anatomic targets.

These inputs 500, 502, 504, 506 may be received by the system 100 in a variety of suitable ways, such as selection by the user from a menu or input by the user via a keypad. The inputs 500, 502, 504, 506 are utilized by the processor 154 to generate an appropriate energy delivery algorithm 152. The algorithm 152 then produces the appropriate energy waveform to provide the desired PEF energy. In some embodiments, the system 100 utilizes these inputs 500, 502, 504, 506 in coordination with various sensors or feedback systems to ensure that the waveform continues to be appropriate throughout the treatment procedure. For example, temperature sensors may be utilized to ensure that any temperature rise does not surpass the maximum allowable temperature rise provided as the second input 502. If readings indicate exceeding the maximum, a warning indication may be provided, a different energy delivery algorithm may be utilized, energy delivery may automatically be ceased or other suitable protocol changes may ensue.

It may be appreciated that any number of inputs 500, 502, 504, 506 may be utilized. Although four different inputs are shown in the above example, one, two, three, four, five, six, seven, eight, nine, ten or more than ten inputs may be utilized. Other inputs may include the type of catheter 102, the type of energy delivery electrode 122, the type of tissue, the thickness of tissue, activation pattern of the energy delivery electrode, the location of other tissues, challenges the doctor is experiencing (e.g., too much cough), proximity of non-target tissues, contact force, local tissue impedance, system impedance, etc. Likewise, if only a subset of the inputs 500, 502, 504, 506 are desired to be selected, default inputs may be used for the remaining inputs. The value of such default inputs may be based on clinical averages, pre-programmed information or calculated based on the inputs provided or other available information. In some embodiments, the inputs may be ranked or weighted so that, for example, a suitable waveform can be generated even if each input is not absolutely conformed to. For example, in some instances, there are primary inputs that are of critical importance and secondary inputs that are desired but not critical. Therefore, if a waveform cannot be generated based on all of the inputs (this may particularly be the case when selecting an algorithm from a pre-determined selection of algorithms) the primary inputs may be prioritized to ensure that at least the best fitting waveform is provided to the user. The ranking or weighting of the inputs may be based on a variety of factors, including anatomical location of the energy delivery, patient specific anatomic variability, safety considerations, other procedural characteristics and/or other information.

As mentioned, the system 100 includes a processor 154 that generates the appropriate energy delivery algorithm 152 with the use of the data storage/retrieval unit 156, among others. The appropriate energy delivery algorithm 152 then produces the desired waveform for PEF energy delivery. The stored information utilized by the processor 154 to generate the algorithms 152 is generated by a series of steps. In one embodiment, the first step is to correlate the effects of the different waveform parameters to treatment aspects, such as the four inputs 500, 502, 504, 506 of FIG. 20. In this embodiment, this is achieved by performing parameter sweeps in a model. A parameter sweep is when one parameter is changed through a range of values while the other parameters are fixed and the effects are observed. Typically, a general baseline of parameters are used as the fixed values. The sweeps are done to a value that achieves the effective lethal electric field thresholds.

It may be appreciated that the model can be the final targeted tissue (e.g. in vivo beating heart), or a substitute model that reflects the same relationships with respect to dose dependency (e.g. another organ, such as thigh, liver, etc., an ex vivo perfused organ model, or a benchtop model, such as a vegetal potato or apple). In some instances, a simple electrode geometry (e.g. needle in a potato immersed in saline bath or bipolar electrode couplet array with both electrodes placed into a potato at a standard distance apart and depth) can be used because the effect of the electrode geometry can be scaled. When using a benchtop model, a physical setup that mimics the energy delivery dynamics of the therapy is desired. For example, testing of a cardiac ablation device used to deliver treatment onto the surface of a target tissue may be simulated by delivering energy to a surface of a potato.

In this embodiment, the tissue is in a bath of fluid with electrical conductivity selected to best approximate the total in vivo environment. For monopolar electrodes, this clinically includes the heart, pericardial sac, lungs, muscle, fat, skin, and other tissues used to conduct the electrical pulse energy between the affector electrode and the distant remote dispersive electrode. It may be appreciated that, in bench models, the dispersive ground electrode may be placed adjacent, below, or in another environment to best represent the electrical PEF energy traveling through the tissue to reach the ground electrode, such that the energy travels through the model in a similar fashion. For bipolar electrodes, this may just be the fluid between the activated regions of the electrode for given bipolar electrode array couplets.

An example of parameters and testing sweeps is provided in Table 3. In this example, the baseline set of parameters included 1000V applied voltage with baseline waveform of a biphasic 100 kHz frequency with 10 cycles for 10 packets delivered at 1 packet per second.

TABLE 3

| Parameter | Range | Step options |
|---|---|---|
| Frequency, kHz | 10 kHz-1000 kHz | 50 kHz |
| Cycles, ct | 1-100 | 5 |
| Packets, ct | 1-100 | 5 |
| Packet Delivery rate, | 0.1-10 | 1 |
| packets per second | | |

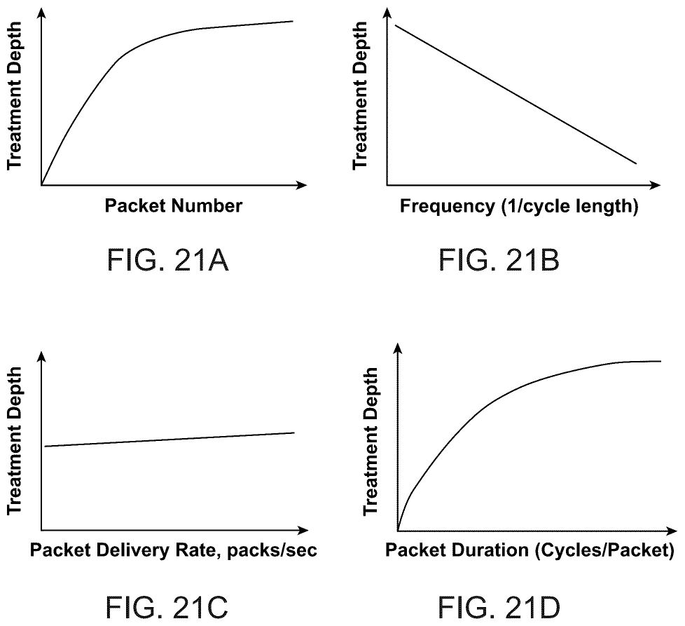

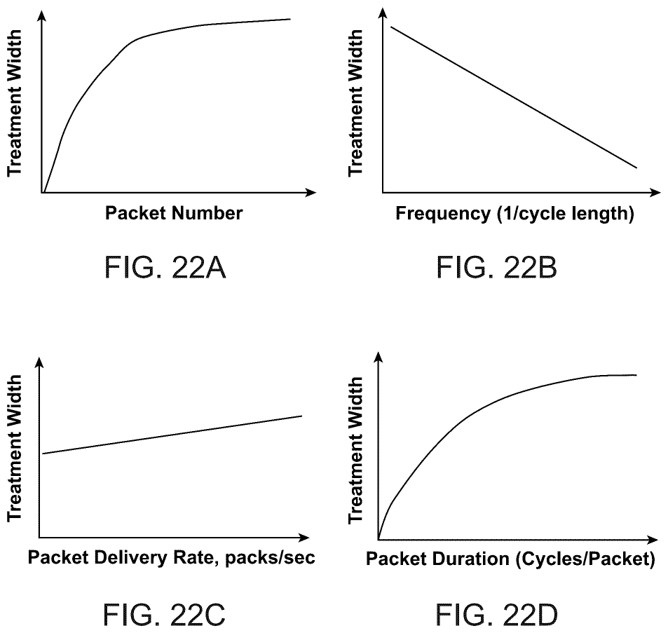

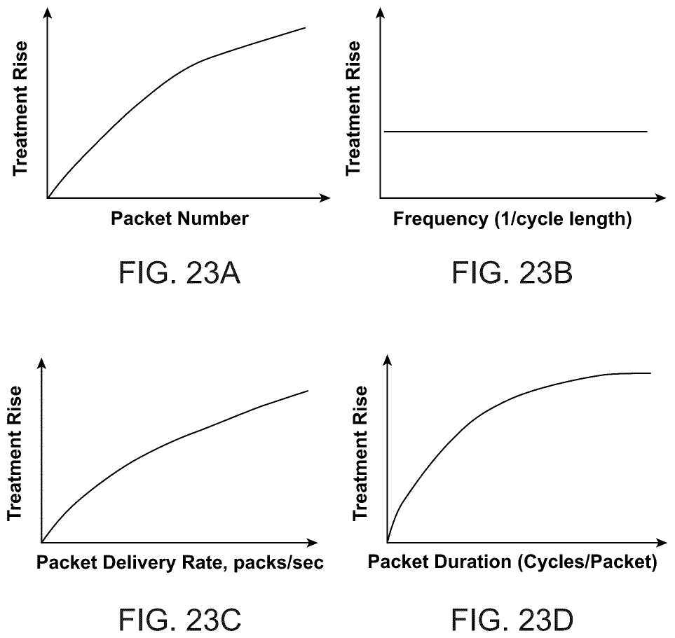

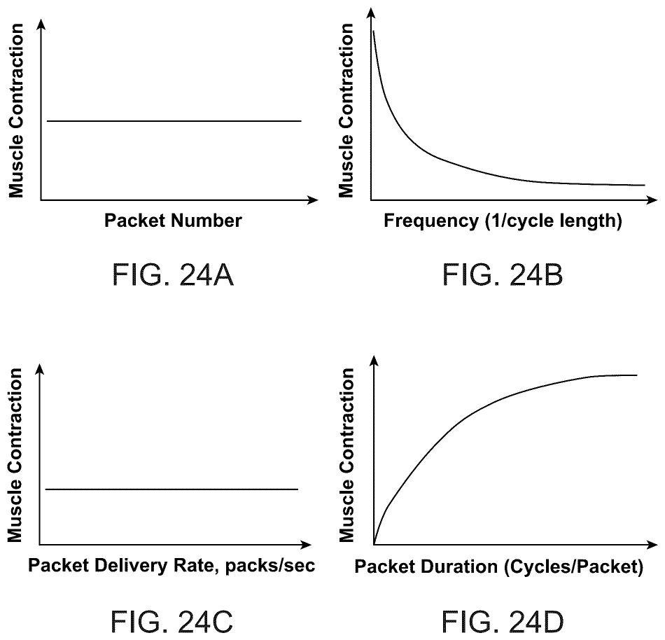

Following treatment delivery with all the parameter combinations, functions are generated to determine how each parameter individually influences treatment effect. In this example, the inputs 500, 502, 504 of FIG. 20 are utilized as the treatment effects of interest. In particular, FIGS. 21A-21D. FIGS. 22A-22D. FIGS. 23A-23D. FIGS. 24A-24D illustrate the individual relationships between the parameters of packet number, frequency, packet delivery rate and packet duration and the inputs 500. 502. 504 from which the functions are mathematically generated. Input 500 regarding lesion dimensions has been split into two measurements, treatment depth (i.e., lesion depth) and treatment width (i.e., lesion width). Input 502 refers to temperature rise and input 504 refers to muscle contraction, gathered via accelerometer (using in vivo studies). In particular. FIG. 21A illustrates the relationship between packet number and treatment depth. FIG. 21B illustrates the relationship between frequency and treatment depth. FIG. 21C illustrates the relationship between packet delivery rate and treatment depth. And. FIG. 21D illustrates the relationship between packet duration and treatment depth. Likewise. FIG. 22A illustrates the relationship between packet number and treatment width. FIG. 22B illustrates the relationship between frequency and treatment width. FIG. 22C illustrates the relationship between packet delivery rate and treatment width. And. FIG. 22D illustrates the relationship between packet duration and treatment width. Further, FIG. 23A illustrates the relationship between packet number and temperature rise. FIG. 23B illustrates the relationship between frequency and temperature rise. FIG. 23C illustrates the relationship between packet delivery rate and temperature rise. And. FIG. 23D illustrates the relationship between packet duration and temperature rise. Still further. FIG. 24A illustrates the relationship between packet number and muscle contraction. FIG. 24B illustrates the relationship between frequency and muscle contraction. FIG. 24C illustrates the relationship between packet delivery rate and muscle contraction. And. FIG. 24D illustrates the relationship between packet duration and muscle contraction.

It may be appreciated that resultant lesion sizes are dependent on the electrode arrangement used in the model. Therefore, a numerical simulation of the electric field strengths for the electrode arrangement is performed to correlate the lesion sizes to electric field strengths.

Following this, an additional numerical simulation, or multiple simulations, may be constructed to represent different treatment delivery physical setups. For example, a focal monopolar catheter electrode may be modeled within the atrial chamber of the heart, placed against myocardium. The electric field distribution of these simulations may be captured and retained. In this way, the applied voltage and lethal electric field threshold functions may be compiled to generate a lookup table or other method of capturing this data.

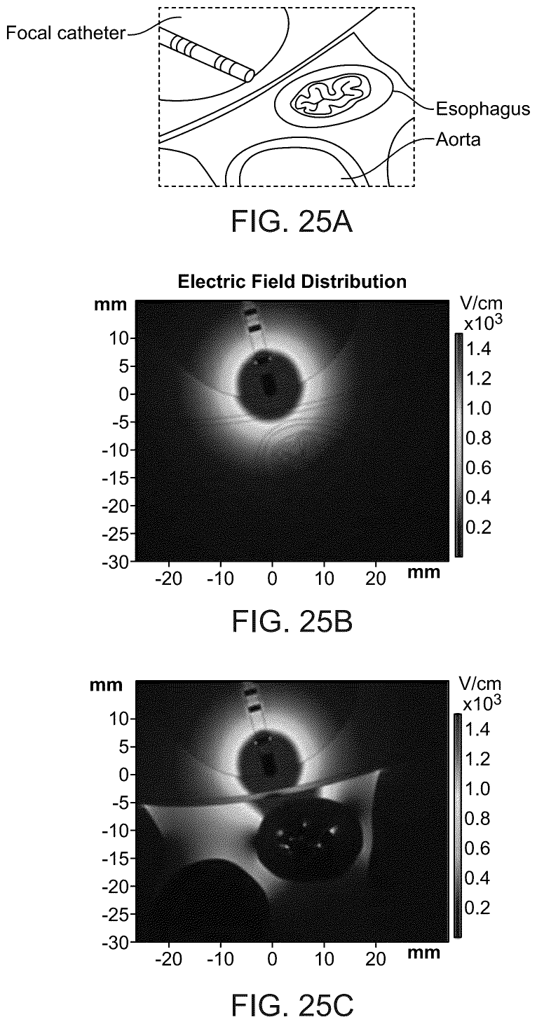

Additional numerical simulations can be made of other electrode arrangements. For example, a focal monopolar catheter electrode may be modeled within the atrial chamber of the heart, placed against myocardium. Using the numerical model, the electric field distribution can be determined in each scenario. This can be captured in a lookup table or other method capturing this data. FIGS. 25A-25C illustrate example electric field distributions for a catheter embodiment delivering PEF energy within the heart.

The electric field data is then calibrated to ensure that the model reflects a clinical scenario. This is achieved by testing a subset of treatment parameter combinations in a clinical or in vivo model. The actual data captured from these studies (accelerometer, treatment effect sizes, etc.) are then used to generate a new baseline electric field threshold.

By these methods, correlations between electric field strength and treatment aspects, such as lesion dimensions, etc. are determined. Therefore, referring back to FIG. 20, when a user inputs particular desired lesion dimensions (input 500), the processor 154 uses the desired lesion dimensions and correlates this to electric field strength thresholds. The processor 154 then uses the electrode arrangement to determine the electric field strengths and determines the electric field strength thresholds that correspond to the desired lesion dimensions. The processor 154 then determine the parameter values to generate the determined electric field strengths thereby generating the desired waveform. These same methods may be used for any of the inputs to determine the desired waveform. Likewise, when combining inputs, the processor 154 takes into account the combined effects of the various parameter influences to select the ultimate parameters leading to the generation of the most suitable waveform. It may be appreciated that the processor 154 may also take into account ranking or weighting of the inputs to generate the most suitable waveform.

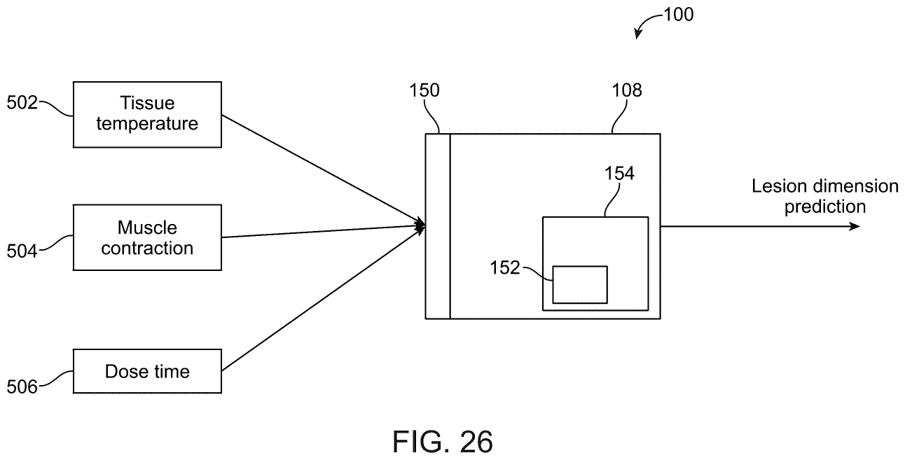

It may be appreciated that the above described methodology may also be used to allow a user to provide inputs to the system 100 and receive a prediction, such as lesion dimensions, temperature rise, muscle contraction, dose time, etc. In one embodiment, illustrated in FIG. 26, the generator 108 includes at least one algorithm 152 that generates a predetermined waveform. The algorithm 152 is modifiable by changing the parameters based on the inputs 502, 504, 506 similar to above. Based on the correlations described above, the user is provided the lesion dimensions. If such lesion dimensions are desirable, the user may proceed to use the new modified waveform. If different dimensions are desired, the user may change one or more the inputs 502, 504, 506, such as in an iterative fashion, until the desired lesion dimensions are achieved. It may be appreciated that inputs and outputs may have a variety of combinations as desired.

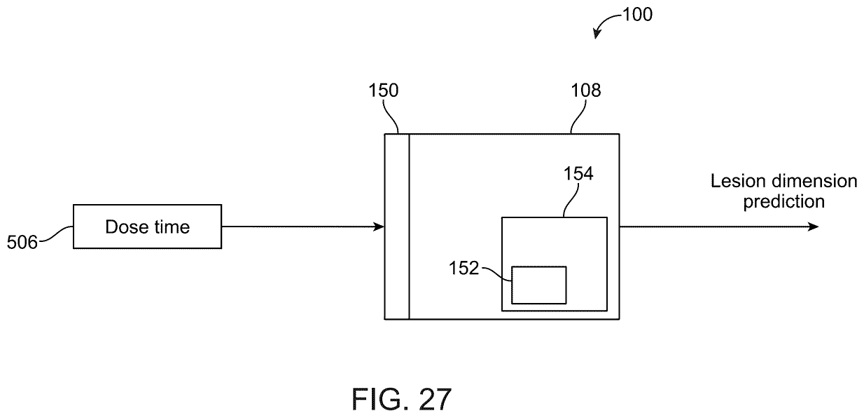

It may be appreciated that the above described methodology may also be used to allow a user to provide a single input to the system 100 and receive a prediction such as lesion dimensions. In one embodiment, illustrated in FIG. 27, the generator 108 includes at least one algorithm 152 that generates a predetermined waveform. In this embodiment, the user provides an input 506, such as dose time. Thus. the user has a desired dose time based on various clinical constraints. Based on the correlations described above, the user is provided the lesion dimensions that result from delivering the predetermined waveform. If such lesion dimensions are desirable, the user may proceed to use the waveform. This assists the user in determining the placement of the lesions, such as to provide a desired level of overlap. This is particularly useful in cardiac applications, such as when generating an electrical blockade.

It may be appreciated that in some embodiments, the system of FIG. 27 is reversed wherein the user provides an input 500 of desired lesion dimension and a predicted dose time is provided by the system 100. When doses are delivered manually, such as by a footswitch, the user may then provide the appropriate dose time or number of doses. When doses are delivered automatically, the user can decide if the dose time is appropriate for the clinical situation and actuate the delivery as desired.

Control over lesion dimensions is desirable when determining lesion spacing. As mentioned previously, continuous lesions can be formed by the repeated application of energy in point-by-point fashion, typically wherein each lesion L overlaps an adjacent lesion L. The desired spacing between the lesions L, and therefore the amount of overlap, depends at least on the size of the lesion L generated. Once a lesion size is known, spacing can be determined by the user either alone or assisted by the system 100. Default or known dimensions of stun zones for each given lesion size may determine how close together the lesions should be placed to ensure a durable continuous lesion. In some embodiments, the system 100 indicates the desired spacing, such as in a numerical readout or in a graphical format, to name a few.

In some embodiments, the system 100 uses local electrophysiologic response to provide dynamic spacing. In such embodiments, a dose of PEF energy is delivered through a catheter having at least one delivery electrode. The same or a different catheter then maps the spatial extent of the voltage reduction. Lesion spacing or PEF application location spacing may be driven by the actual voltage reduction observed in response to the dose and electrode configuration. In some embodiments, spacing takes the form of a fixed overlap and in other embodiments spacing is user-selectable, such as based on a desired safety factor. This can be performed manually or automated through software that interprets the intra-procedure maps and post application electrogram voltage changes.

It may also be appreciated that pre-procedure imaging data may be used to dynamically control safety factor/spacing. For tissue areas known or suspected to be thicker, the dynamic spacing recommendation would be smaller to ensure transmural lesions. For example, intracardiac echography may be used to measure the thickness of the tissue adjacent to the electrode. This could be measured manually and input into the system, or the ultrasound could communicate directly with the generator to feed any thickness data. In another embodiment, an ultrasound transducer may be incorporated onto the catheter electrode, and it may measure tissue thickness locally or throughout the organ chamber prior to energy delivery, or at the site of intended energy delivery immediately prior to energy delivery. In another embodiment, 3D magnetic resonance (MR)-ultrasound (US) fusion or MR-mapping system fusion may be used correlate the anatomy with a targeted tissue thickness. In this way. MR may be used to visualize and segment the chamber wall thickness in 3D. This data may be married to the mapping or ultrasound data via a series of fiducial landmarks to orient them. The thickness data may then be projected onto the electroanatomical mapping system, which may be done directly for MR-mapping fusion, or with the interface of an ultrasound machine for the MR-US fusion. This thickness data may be used to establish the treatment dose parameters to use in the region, or to assist the clinician in setting the parameters. In another approach, optical coherence tomography may be used to measure tissue thickness. In each instance of these described embodiments, the thickness data may be generated and interpreted in real-time at the site of interest for where the energy will be delivered, is being delivered, or has been delivered. The data may also be used to generate an entire targeted reconstruction of thicknesses prior to energy delivery. whereby measurements are not taken in real-time immediately prior to energy delivery. In some instances, the 3D mapping may be performed retrospectively after energy delivery to determine any regions that may be undertreated. This may be done with knowledge of the dosing being used, or with the PEF or mapping system logging each specific dose at each specific activation, and recording this data to compare with the thickness data later to evaluate any regions of undertreatment.

In some embodiments, the generator 108 generates “smart tag” information that is incorporated into any sort of display of the generated lesions. In cardiac applications, the smart tag may appear on the display of a mapping system, or the smart tag may be stored or displayed through the generator, or other data collection systems used to support the ablation procedure. In some embodiments, the smart tags include information such as treatment depth and width, predicted thermal rise, predicted muscle stimulation, and other relevant parameters of interest to the operator when performing or reviewing a treatment.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B.” “B but not A.” and “A and B.” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims. Also, in the above Detailed Description. various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A system for generating a lesion in tissue comprising:
a generator electrically couplable to a treatment catheter, wherein the generator includes a processor that generates at least one algorithm configured to provide an electric signal of pulsed electric field energy deliverable through a delivery electrode of the treatment catheter, wherein the processor generates the algorithm based on at least one of one or more inputs by a user.

2. A system as in claim 1, wherein the processor generates the algorithm by dynamically generating the algorithm based on the one or more inputs in real time.

3. A system as in claim 1, wherein the processor generates the algorithm by selecting the algorithm from a group of algorithms based on the one or more inputs.

4. A system as in claim 1, wherein the one or more inputs comprises a desired dimension of the lesion.

5. A system as in claim 1, wherein the one or more inputs comprises a threshold for a temperature rise.

6. A system as in claim 1, wherein the one or more inputs comprises a threshold for muscle contraction.

7. A system as in claim 1, wherein the one or more inputs comprises a threshold dose time.

8. A system as in claim 1, wherein the one or more inputs comprises a plurality of the one or more inputs and wherein the processor utilizes the plurality of the one or more inputs and based on a ranking or weighting of the plurality of the one or more inputs.

9. A system as in claim 1, wherein the processor utilizes the one or more inputs and at least one default value to generate the algorithm.

10. A system as in claim 1, wherein the processor correlates the one or more inputs to electric field strength thresholds.

11. A system as in claim 10, wherein the processor determines the electric field strengths of the at least one delivery electrode.

12. A system as in claim 11, wherein the processor correlates the electric field strengths to the electric field strength thresholds.

13. A system as in claim 1, wherein the processor selects at least one parameter of the at least one algorithm to generate the electric signal.

14. A system as in claim 1, wherein the processor modifies the algorithm based on tissue thickness.

15. A system as in claim 14, wherein tissue thickness is determined by ultrasound, magnetic resonance or fluoroscopy.

16. A system as in claim 1, wherein the processor generates a prediction of an outcome of the treatment based on at least one of one or more inputs by a user.

17. A system as in claim 16, wherein the prediction comprises at least one lesion dimension.

18. A system as in claim 17, wherein the at least one lesion dimension comprises lesion depth.

19. A system as in claim 17, wherein the at least one lesion dimension comprises lesion width.

20. A system as in claim 16, wherein the one or more inputs comprises a single input, wherein the single input comprises dose time.
